(19) United States
(12) Patent Application Publication
(10) Pub. No.: US 2013/0005743 A1
(43) Pub. Date: Jan. 3, 2013

(54) New positive allosteric modulators of nicotinic acetylcholine receptor

(75) Inventors: Jørgen Eskildsen, Kobenhavn (DK); Christian Wenzel Tornøe, Lyngby (DK)

(73) Assignee: H. Lundbeck A/S, Valby-Copenhagen (DK)

(21) Appl. No.: 13/537,129

(22) Filed: Jun. 29, 2012

Related U.S. Application Data
(60) Provisional application No. 61/504,355, filed on Jul. 5, 2011.
(60) Provisional application No. 61/503,633, filed on Jul. 1, 2011.

(51) Int. Cl. A61K 31/437; C07D 471/04; A61P 25/18; A61P 25/00; A61P 29/00; A61P 25/16; A61P 25/08; A61P 25/24; A61P 3/10; A61P 3/04; A61K 31/506; A61P 25/28

(52) U.S. Cl. 514/256; 514/303; 544/322; 546/120

(57) ABSTRACT

The present invention relates to compounds useful in therapy, to compositions comprising said compounds, and to methods of treating diseases comprising administration of said compounds. The compounds referred to are positive allosteric modulators (PAMs) of the nicotinic acetylcholine α7 receptor.

### FIELD OF THE INVENTION

The present invention relates to compounds useful in therapy, to compositions comprising said compounds, and to methods of treating diseases comprising administration of said compounds. The compounds referred to are positive allosteric modulators (PAMs) of the nicotinic acetylcholine α7 receptor.

### BACKGROUND OF THE INVENTION

Nicotinic acetylcholine receptors (nAChRs) belong to the super family of ligand gated ionic channels, and gate the flow of cations including calcium. The nAChRs are endogenously activated by acetylcholine (ACh) and can be divided into nicotinic receptors of the neuromuscular junction and neuronal nicotinic receptors (NNRs). The NNRs are widely expressed throughout the central nervous system (CNS) and the peripheral nervous system (PNS). The NNRs have been suggested to play an important role in CNS function by modulating the release of many neurotransmitters, for example, ACh, norepinephrine, dopamine, serotonin, and GABA, among others, resulting in a wide range of physiological effects.

Seventeen subunits of nAChRs have been reported to date, which are identified as α2-α10, β1-β4, γ, δ and ε. From these subunits, nine subunits, α2 through α7 and β2 through β4, prominently exist in the mammalian brain. Many functionally distinct nAChR complexes exist, for example five α7 subunits can form a receptor as a homomeric functional pentamer or combinations of different subunits can form heteromeric receptors such as α4β2 and α3β4 receptors (Gotti, C. et al., Prog. Neurobiol., 2004, 74: 363-396; Gotti, C., et al. Biochemical Pharmacology, 2009, 78: 703-711)

The homomeric α7 receptor is one of the most abundant NNRs, along with α4β2 receptors, in the brain, wherein it is heavily expressed in the hippocampus, cortex, thalamic nuclei, ventral tegmental area and substantia nigra (Broad, L. M. et al., Drugs of the Future, 2007, 32(2): 161-170, Poorthuis, R. B., Biochem Pharmacol. 2009 1; 78(7):668-76).

The role of α7 NNR in neuronal signalling has been actively investigated. The α7 NNRs have been demonstrated to regulate interneuron excitability and modulate the release of excitatory as well as inhibitory neurotransmitters. In addition, α7 NNRs have been reported to be involved in neuroprotective effects in experimental models of cellular damage (Shimohama, S., Biol Pharm Bull. 2009, 32(3):332-6). Studies have shown that α7 subunits, when expressed recombinant in-vitro, activate and desensitize rapidly, and exhibit relatively higher calcium permeability compared to other NNR combinations (Papke R. L., et al., J Pharmacol Exp Ther. 2009, 329(2):791-807).

The NNRs, in general, are involved in various cognitive functions, such as learning, memory and attention, and therefore in CNS disorders, i.e., Alzheimer's disease (AD), Parkinson's disease (PD), attention deficit hyperactivity disorder (ADHD), Tourette's syndrome, schizophrenia, bipolar disorder, pain and tobacco dependence (Keller, J. J. et al., Behav. Brain Res. 2005, 162: 143-52; Haydar; S, N. et al., Curr Top Med. Chem. 2010; 10(2):144-52).

The α7 NNRs in particular, have also been linked to, cognitive disorders including, for example, ADHD, autism spectrum disorders, AD, mild cognitive impairment (MCI), age associated memory impairment (AAMI) senile dementia; frontotemporal lobar degeneration, HIV associated dementia (HAD), HIV associated cognitive impairment (HIV-CI), Pick's disease, dementia associated with Lewy bodies, cognitive impairment associated with Multiple Sclerosis, Vascular Dementia, cognitive impairment in Epilepsy, cognitive impairment associated with fragile X, cognitive impairment associated with Friedreich's Ataxia, and dementia associated with Down's syndrome, as well as cognitive impairments associated with schizophrenia. In addition, α7-NNRs have been shown to be involved in the neuroprotective effects of nicotine both in vitro (Jonnala, R. B. et al., J. Neurosci. Res., 2001, 66: 565-572) and in vivo (Shimohama, S., Brain Res., 1998, 779: 359-363) as well as in pain signalling. More particularly, neurodegeneration underlies several progressive CNS disorders, including, but not limited to, AD, PD, amyotrophic lateral sclerosis, Huntington's disease, dementia with Lewy bodies, as well as diminished CNS function resulting from traumatic brain injury. For example, the impaired function of α7 NNRs by beta-amyloid peptides linked to AD has been implicated as a key factor in development of the cognitive deficits associated with the disease (Liu, Q.-S. et al., PNAS, 2001, 98: 4734-4739). Thus, modulating the activity of α7 NNRs demonstrates promising potential to prevent or treat a variety of diseases indicated above, such as AD, other dementias, other neurodegenerative diseases, schizophrenia and neurodegeneration, with an underlying pathology that involves cognitive function including, for example, aspects of learning, memory, and attention (Thomsen M. S. et al., Curr. Pharm. Des. 2010 January; 16(3):323-43; Olincy, A., et al., Arch Gen Psychiatry. 2006, 63(6):630-8; Deutsch, S. I., Clin Neuropharmacol. 2010, 33(3):114-20; Feuerbach, D., Neuropharmacology. 2009, 56(1): 254-63)

The NNR ligands, including α7 ligands, have also been implicated in weight control, diabetis inflammation, obsessive-compulsive disorder (OCD), angiogenesis and as potential analgesics (Marrero, M. B. et al., J. Pharmacol. Exp. Ther. 2010, 332(1):173-80; Vincler, M., Exp. Opin. Invest. Drugs, 2005, 14 (10): 1191-1 198; Rosas-Ballina, M., J. Intern Med. 2009 265(6):663-79; Arias, H. R., Int. J. Biochem. Cell Biol. 2009, 41(7):1441-51; Tizabi Y, Biol Psychiatry. 2002 Jan. 15; 51(2):164-71).

Nicotine is known to enhance attention and cognitive performance, reduced anxiety, enhanced sensory gating, and analgesia and neuroprotective effects when administered. Such effects are mediated by the non-selective effect of nicotine at multiple nicotinic receptor subtypes. However, nicotine also exerts adverse events, such as cardiovascular and gastrointestinal problems (Karaconji I B, et al., Arh Hig Rada Toksikol. 2005 56(4):363-71). Consequently, there is a need to identify subtype-selective compounds that retain the beneficial effects of nicotine, or an NNR ligand, while eliminating or decreasing adverse effects.

Examples of reported NNR ligands are α7 NNR agonists, such as DMXB-A, SSR180711 and ABT-107, which have shown some beneficial effects on cognitive processing both in rodents and humans (H312: 1213-22; Olincy, A., et al., Arch Gen Psychiatry. 2006 63(6):630-8; Pichat, P., et al., Neuropsychopharmacology. 2007 32(1):17-34; Bitnet R. S., J Pharmacol Exp Ther. 2010 1; 334(3):875-86). In addition, modulation of α7 NNRs have been reported to improve negative symptoms in patients with schizophrenia (Freedman R, et al., Am J Psychiatry. 2008 165(8):1040-7).

Despite the beneficial effects of NNR ligands, it remains uncertain whether chronic treatment with agonists affecting NNRs may provide suboptimal benefit due to sustained activation and desensitization of the NNRs, in particular the α7 NNR subtype. In contrast to agonists, administering a positive allosteric modulator (PAM) can reinforce endogenous cholinergic transmission without directly stimulating the target receptor. Nicotinic PAMs can selectively modulate the activity of ACh at NNRs, preserving the activation and deactivation kinetics of the receptor. Accordingly, α7 NNR-selective PAMs have emerged (Faghih R, Recent Pat CNS Drug Discov. 2007, 2(2):99-106).

Consequently, it would be beneficial to increase α7 NNR function by enhancing the effect of the endogenous neurotransmitter acetvlcholine-Via PAMs. This could reinforce the endogenous cholinergic, neurotransmission without directly activating α7 NNRs, like agonists. Indeed, PAMs for enhancing channel activity have been proven clinically successful for GABAa receptors where benzodiazepines and barbiturates, behave as PAMs acting at distinct sites (Hevers, W. et al., Mol. Neurobiol., 1998, 18: 35-86).

To date, only a few NNR PAMs are known, such as 5-hydroxyindole (5-HI), ivermectin, galantamine, and SLURP-1, a peptide derived from acetylcholinesterase (AChE). Genistein, a kinase inhibitor was also reported to increase α7 responses. PNU-120596, a urea derivative, was reported to increase the potency ACh as well as improve auditory gating deficits induced by amphetamine in rats. Also, NS1738, JNJ-1930942 and compound 6 have been reported to potentiate the response of ACh and exert beneficial effect in experimental models of sensory and cognitive processing in rodents. Other NNR PAMs include derivatives of quinuclidine, indole, benzopyrazole, thiazole, and benzoisothiazoles (Hurst, R. S. et al., J. Neurosci., 2005, 25: 4396-4405; Faghih, R., Recent Pat CNS Drug Discov. 2007, 2(2):99-106; Timmermann, D. B., J Pharmacol Exp Ther. 2007, 323(1):294-307; Ng, H. J. et al., Proc. Natl. Acad. Sci. USA. 2007, 104(19):8059-64; Dinklo T, J. Pharmacol Exp Ther. 2011 336(2):560-74.).

Of particular examples WO 01/32619 recites that compounds of the following core structure are PAMs of the α7 NNR

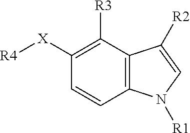

and WO 2009/100294 discloses compounds with the core structure

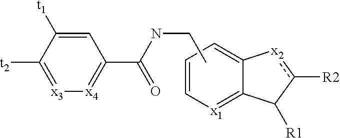

which are said to be PAMs of the α7 NNR.

The α7 NNR PAMs presently known generally demonstrate weak activity, have a range of non-specific effects, or can only achieve limited access to the central nervous system where α7 NNRs are abundantly expressed. Accordingly, it would be beneficial to identify and provide new PAM compounds of α7 NNRs and compositions for treating diseases and disorders wherein α7 NNRs are, involved. It would further be particularly beneficial if such compounds can provide improved efficacy of treatment while reducing adverse effects associated with compounds targeting neuronal nicotinic receptors by selectively modulating α7 NNRs.

### SUMMARY OF THE INVENTION

The objective of the present invention is to provide compounds that are positive allosteric modulators (PAMs) of the nicotinic acetylcholine receptor subtype α7.

The compounds of the present invention are defined by formula III below:

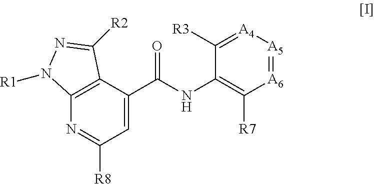

wherein A4 is C—R4 or N, A5 is C—R5 or N and A6 is C—R6 or N, provided that at least one of A4, A5 or A6 is N and no more than two of A4, A5 and A5 is N;
R1 is phenyl or heteroaryl; wherein said phenyl or heteroaryl is optionally substituted with one or more substituents R11, wherein each R11 is individually selected from C₁₋₆alkyl, halogen, hydroxy, haloC₁₋₆alkyl, C₁₋₆alkoxy, haloC₁₋₆alkoxy, cyano, —S(O)₂NH₂ and —NR12R13, wherein R12 and R13 independently represent hydrogen or C₁₋₆alkyl;
R2 is selected from H, C₁₋₆alkyl, C₂₋₆alkenyl, C₂₋₆alkynyl, C₁₋₆alkoxy, halogen and cyano;
R3, R4, R5, R6 and R7 are selected independently of each other from H, C₁₋₆alkyl, C₂₋₆alkenyl, C₂₋₆alkynyl, C₁₋₆alkoxy, halogen, cyano, and —NR9R10, wherein R9 and R10 independently represent hydrogen, C₁₋₆alkyl, C₂₋₅alkenyl, C₂₋₆alkynyl, haloC₁₋₆alkyl or phenyl;
R8 is selected from H, C₁₋₆alkyl, C₂alkenyl, C₂₋₆alkynyl, C₁₋₆alkoxy, halogen, cyano and phenyl; and pharmaceutically acceptable salts thereof.

In one embodiment, the invention relates to a compound according to formula [I], and pharmaceutically acceptable salts thereof, for use as a medicament.

In one embodiment, the invention relates to a compound according to formula [I], and pharmaceutically acceptable salts thereof, for use in therapy.

In one embodiment; the invention relates to a compound according to formula [I], and pharmaceutically acceptable salts thereof, for use in the treatment of a disease or disorder selected from Psychosis; Schizophrenia; cognitive disorders; cognitive impairment associated with schizophrenia; Attention Deficit Hyperactivity Disorder (ADHD): autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.

In one embodiment, the invention relates to a pharmaceutical composition comprising a compound according to formula [I] and pharmaceutically acceptable salts thereof, and one or more pharmaceutically acceptable carrier or excipient.

In one embodiment, the invention relates to a kit comprising a compound according to formula [I], and pharmaceutically acceptable salts thereof, together with a compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.

In one embodiment, the invention relates to a method for the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); obsessive-compulsive-disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain, which method comprises the administration of a therapeutically effective amount of a compound according to formula [I], and pharmaceutically acceptable salts thereof.

In one embodiment, the invention relates to the use of a compound according to formula [I], and pharmaceutically acceptable salts thereof, for the manufacture of a medicament for the treatment of a disease or disorder selected from Psychosis; Schizophrenia; cognitive disorders; cognitive impairment associated with schizophrenia; Attention Deficit. Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.

### DEFINITIONS

In the present context, “optionally substituted” means that the indicated moiety may or may not be substituted, and when substituted is mono-, di-, or tri-substituted, such as with 1, 2 or 3 substituents. In some instances, the substituent is independently selected from the group consisting of C₁₋₆alkyl, C₂₋₆alkenyl, C₂₋₆alkynyl, phenyl, C₁₋₆alkoxy, hydroxy, halogen and oxo. It is understood that where no substituents are indicated for an “optionally substituted” moiety, then the position is held by a hydrogen atom.

In the present context, “alkyl” is intended to indicate a straight, branched and/or cyclic saturated hydrocarbon. In particular “C₁₋₆alkyl” is intended to indicate such hydrocarbon having 1, 2, 3, 4, 5 or 6 carbon atoms. Examples of C₁₋₆alkyl include methyl, ethyl, propyl, butyl, pentyl, hexyl, cyclopropyl, cyclobutyl, cyclopentyl, cyclohexyl, methylcyclopropyl, 2-methyl-propyl and tert-butyl.

In the present context, “alkenyl” is intended to indicate a non-aromatic, straight, branched and/or cyclic hydrocarbon comprising at least one carbon-carbon double bond. In particular “C₂₋₆alkenyl” is intended to indicate such hydrocarbon having 2, 3, 4, 5 or 6 carbon atoms. Examples of C₂₋₆alkenyl include ethenyl, 1-propenyl, 2-propenyl, 1-butenyl, 2-butenyl and 3-butenyl and cyclohexenyl.

In the present context, “alkynyl” is intended to indicate a non-aromatic, straight, branched and/or cyclic hydrocarbon comprising at least one carbon-carbon triple bond and optionally also one or more carbon-carbon double bonds. In particular “C₂₋₆alkynyl” is intended to indicate such hydrocarbon having 2, 3, 4, 5 or 6 carbon atoms. Examples of C₂₋₆alkynyl include ethynyl, 1-propynyl, 2-propynyl, 1-butynyl, 2-butynyl, 3-butynyl and 5-but-1-en-3-ynyl.

In the present context, “hydroxy” is intended to indicate —OH.

In the present context, “alkoxy” is intended to indicate a moiety of the formula —OR′, wherein R′ indicates alkyl as defined above. In particular “C₁₋₆alkoxy” is intended to indicate such moiety wherein the alkyl part has 1, 2, 3, 4, 5 or 6 carbon atoms. Examples of “C₁₋₆alkoxy” include methoxy, ethoxy, n-butoxy and tert-butoxy.

In the present context, the terms “halo” and “halogen” are used interchangeably and refer to fluorine, chlorine, bromine or iodine.

In the present context, “haloalkyl is intended to indicate an alkyl as defined above substituted with one or more halogens. In particular, haloC₁₋₆alkyl” is intended to indicate a moiety wherein the alkyl part has 1, 2, 3, 4, 5 or 6 carbon atoms. One example of haloalkyl is trifluoromethyl.

In the present context, “haloalkoxy” is intended to indicate an alkoxy as defined above substituted with one or more halogens. In particular, “haloC₁₋₆alkoxy” is intended to indicate a moiety wherein the alkyl part has 1, 2, 3, 4, 5 or 6 carbon atoms. Particular mention is made of difluoromethoxy and trifluoromethoxy.

In the present context, “alkylsulfonyl” is intended to indicate —S(O)₂alkyl In particular C₁₋₆alkylsulfonyl is intended to indicate such a moiety wherein the alkyl part has 1, 2, 3, 4, 5 or 6 carbon atoms. Particular mention is made of methylsulfonyl.

In the present context, the term “cyano” indicates the group —C≡N, which consists of a carbon atom triple-bonded to a nitrogen atom.

In the present context, a “heteroaryl” is intended to indicate a 5 or 6 membered aromatic monocyclic ring containing 1 to 5 carbon atoms and one or more heteroatoms selected from oxygen, nitrogen and sulfur. Examples of heteroaryls of the present invention include 6-membered heteroaryls such as pyridinyl, pyrazinyl, pyridazinyl and pyrimidinyl and 5-membered heteroaryls such as oxathiazolyl, dioxazolyl, dithiazolyl, oxadiazolyl, thiadiazolyl, isoxazolyl, oxazolyl, isothiazolyl, thiazolyl, imidazolyl, pyrazolyl, pyrrolyl, furanyl and thiophenyl. An “N-containing heteroaryl” indicates a 5 or 6 membered aromatic system, wherein at least one ring atom is a nitrogen.

In the present context, when a heteroaryl is optionally substituted with one or more substituents it means that said heteroaryl may or may not be substituted on its carbon atoms, and when substituted it is substituted e.g. with 1, 2 or 3 substituents. In a preferred embodiment an optionally substituted heteroaryl is either not substituted or substituted with one substituent.

In the present context, when a phenyl is optionally substituted with one or more substituents it means that said phenyl may or may not be substituted, and when substituted it is substituted e.g. with 1, 2 or 3 substituents. In a preferred embodiment an optionally substituted phenyl is either not substituted or substituted with one substituent. In the present context, when a phenyl is substituted with one substituent said substituent can be located in the ortho, meta or para position of said phenyl.

In the present context, “ring atom” is intended to indicate the atoms constituting a ring, and ring atoms are selected from C, N, O and S. As an example, benzene and toluene both have 6 carbons as ring atoms whereas pyridine has 5 carbons and 1 nitrogen as ring atoms.

In the present context, “heteroatom” means a nitrogen, oxygen or sulfur atom.

In the present context, pharmaceutically acceptable salts include pharmaceutical acceptable acid addition salts, pharmaceutically acceptable metal salts, ammonium and alkylated ammonium salts. Acid addition salts include salts of inorganic acids as well as organic acids.

Examples of suitable inorganic acids include hydrochloric, hydrobromic, hydroiodic, phosphoric, sulfuric, sulfamic, nitric acids and the like.

Examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutical acceptable inorganic or organic acid addition salts include the pharmaceutically acceptable salts listed in Berge, S. M. et al., J. Pharm. Sci. 1977, 66, 2, which is incorporated herein by reference.

Examples of metal salts include lithium, sodium, potassium, magnesium salts and the like.

Examples of ammonium and alkylated ammonium salts include ammonium, methyl-, dimethyl-, trimethyl-, ethyl-, hydroxyethyl-, diethyl-, n-butyl-, sec-butyl-, tert-butyl-, tetramethylammonium salts and the like.

In the present context, pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier may include any sustained release material known in the art, such as glyceryl monostearate or glyceryl distearate, alone or mixed with a wax.

In the present context, the term “therapeutically effective amount” of a compound means an amount sufficient to cure, alleviate or partially arrest the clinical manifestations of a given disease and its complications in a therapeutic intervention comprising the administration of said compound. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

In the present context, the term “treatment” and “treating” means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. Nonetheless, prophylactic (preventive) and therapeutic (curative) treatments are two separate aspects of the present invention. The patient to be treated is preferably a mammal, in particular a human being.

In the present context, the term “cognitive disorders” is intended to indicate disorders characterized by abnormalities in aspects of perception, problem solving, language, learning, working memory, memory, social recognition, attention and pre-attentional processing, such as by not limited to Attention Deficit Hyperactivity Disorder (ADHD), autism spectrum disorders, Alzheimer's disease (AD), mild cognitive impairment (MCI), age associated memory impairment (AAMI), senile dementia, vascular dementia, frontotemporal lobe dementia, Pick's disease, dementia associated with Lewy bodies, and dementia associated with Down's syndrome, cognitive impairment associated with Multiple Sclerosis, cognitive impairment in epilepsy, cognitive impairment associated with fragile X, cognitive impairment associated with neurofibromatosis, cognitive impairment associated with Friedreich's Ataxia, progressive supranuclear palsy (PSP), HIV associated dementia (HAD), HIV associated cognitive impairment (HIV-CI), Huntington's Disease, Parkinson's disease (PD), traumatic brain injury, epilepsy, post-traumatic stress, Wernicke-Korsakoff syndrome (WKS), post-traumatic amnesia, cognitive deficits associated with depression as well as cognitive impairment associated with schizophrenia.

The cognitive enhancing properties of a compound can be assessed e.g. by the attentional set-shifting paradigm which is an animal model allowing assessment of executive functioning via intra-dimensional (ID) versus extra-dimensional (ED) shift discrimination learning. The study can be performed by testing whether the compound is attenuating “attentional performance impairment” induced by subchronic PCP administration in rats as described by Rodefer; J. S. et al., Eur. J. Neurosci. 21:1070-1076 (2005).

In the present context, the term “autism spectrum disorders” is intended to indicate disorders characterized by widespread abnormalities of social interactions and verbal and non-verbal communication, as well as restricted interests, repetitive behavior and attention, such as by not limited to autism, Asperger syndrome, Pervasive Developmental Disorder Not Otherwise Specified (PDD-NOS), Rett syndrome, Angelmann syndrome, fragile X, DiGeorge syndrome and Childhood Disintegrative Disorder.

In the present context, the term “inflammatory disorders” is intended to indicate disorders characterized by abnormalities in the immune system such as by not limited to, allergic reactions and myopathies resulting in abnormal inflammation as well as non-immune diseases with etiological origins in inflammatory processes are thought to include by not be limited to cancer, atherosclerosis, osteoarthritis, rheumatoid arthritis and ischaemic heart disease.

### DETAILED DESCRIPTION OF THE INVENTION

The present inventors have found that certain new compounds are positive allosteric modulators (PAMs) of NNRs, and as such may be used in the treatment of various diseases and disorders.

PAMs of NNRs may be dosed in combination with other drugs in order to achieve more efficacious treatment in certain patient populations. An α7 NNR PAM may act synergistically with another drug, this has been described in animals for the combination of compounds affecting nicotinic receptors, including α7 NNRs and D2-antagonism (Wiker, C., Int. J. Neuropsychopharmacol. 2008 September; 11(6):845-50).

Thus, compounds of the present invention may be useful treatment in the combination with another drug e.g. selected from acetylcholinesterase inhibitors, glutamate receptor antagonists, dopamine transport inhibitors, noradrenalin transport inhibitors, D2 antagonists, D2 partial agonists, PDE10 antagonists, 5-HT2A antagonists, 5-HT6 antagonists and KCNQ antagonists, lithium, sodium channel blockers, GABA signalling enhancers.

In one embodiment, compounds of the present invention are used for treatment of patients who are already in treatment with another drug selected from the list above. In one embodiment, compounds of the present invention are adapted for administration simultaneous with said other drug. In one embodiment compounds of the present invention are adapted for administration sequentially with said other drug. In one embodiment, compounds of the present invention are used as the sole medicament in treatment of a patient. In one embodiment, compounds of the present invention are used for treatment of patients who are not already in treatment with another drug selected from the list above.

### EMBODIMENTS ACCORDING TO THE INVENTION

In the following, embodiments of the invention are disclosed. The first embodiment is denoted E1, the second embodiment is denoted E2 and so forth.

E1. A compound according to formula [I]

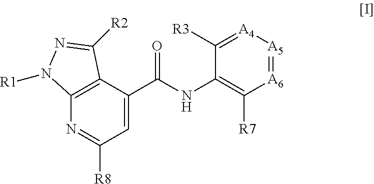

wherein A4 is C—R4 or N, A5 is C—R5 or N and A6 is C—R6 or N, provided that at least one of A4, A5 or A6 is N and no more than two of A4, A5 and A6 is N;
R1 is phenyl or heteroaryl; wherein said phenyl or heteroaryl is optionally substituted with one or more substituents R11, wherein'each R11 is individually selected from C₁₋₆alkyl, halogen, hydroxy, C₁₋₆alkoxy, haloC₁₋₆alkoxy, cyano, C₁₋₆alkylsulfonyl, —S(O)₂NH₂ and —NR12R13, wherein R12 and R13 independently represent hydrogen or C₁₋₆alkyl;
R2 is selected from H, C₂₋₆alkenyl, C₂₋₆alkynyl, C₁₋₆alkoxy, halogen and cyano;
R3, R4, R5, R6 and R7 are selected independently of each other from H, C₁₋₆alkyl, C₂₋₆alkenyl, C₂₋₆alkynyl, C₁₋₆alkoxy, halogen, cyano, and —NR9R10, wherein R9 and R10 independently represent hydrogen, C₁₋₆alkyl, C₂₋₆alkenyl, C₂₋₆alkynyl, haloC₁₋₆alkyl or phenyl;
R8 is selected from H, C₁₋₆alkyl, C₂₋₆alkenyl, C₂₋₆alkynyl, C₁₋₆alkoxy, halogen, cyano and phenyl;
and pharmaceutically acceptable salts thereof.
E2. The compound according to embodiment 1, wherein each R11 is individually selected from methyl, trifluoromethyl, fluorine, hydroxy, methoxy, difluoromethoxy, trifluoromethoxy, cyano, methylsulfonyl, —S(O)₂NH₂ and —N(CH₃)₂;
R2 is selected from H, methyl and methoxy;
R3, R4, R5, R6 and R7 are selected independently of each other from H, methyl, methoxy, cyano and N(CH₃)₂;
R8 is selected from H, methyl and phenyl.
E3. The compound according to any of embodiments 1-2, wherein only one of A4, A5 or A6 is N.
E4. The compound according to any of embodiments 1-3, wherein A4 is N, A5 is C—R5 and A6 is C—R6.
E5. The compound according to any of embodiments 1-3, wherein A5 is N and A4 is C—R4 and A6 is C—R6.
E6. The compound according to any of embodiments 1-3, wherein A6 is N and A4 is C—R4 and A5 is C—R5.
E7. The compound according to any of embodiments 1-2, wherein two of A4, A5 or A6 are N
E8. The compound according to embodiment 7, wherein A4 and A5 are both N and A6 is C—R6.
E9. The compound according to embodiment 7, wherein A4 and A6 are both N and A5 is C—R5.
E10. The compound according to embodiment 7, wherein A5 and A6 are both N and A4 is C—R4.
E11. The compound according to any of embodiments 1-10, wherein R1 represents a phenyl optionally substituted with one or more R11.
E12. The compound according to any of embodiments 1-10, wherein R1 represents a 6-membered heteroaryl optionally substituted with one or more R11.
E13. The compound according to embodiment 12, wherein R1 represents an N-containing 6-membered heteroaryl optionally substituted with one or more R11.
E14. The compound according to any of embodiments 12-13, wherein R1 is selected from pyridinyl, pyrazinyl, pyridazinyl or pyrimidinyl optionally substituted with one R11.
E15. The compound according to embodiment 14, wherein R1 represents pyridinyl optionally substituted with one R11.
E16. The compound according to any of embodiments 1-10, wherein R1 represents a 5-membered heteroaryl optionally substituted with one or more R11.
E17. The compound according to any of embodiments 1-16, wherein R1 is optionally substituted with one R11.
E18. The compound according to any of embodiments 1-11, wherein R1 is a phenyl optionally substituted with one R11, wherein said R11 is located in the para position of said phenyl.
E19. The compound according to any of embodiments 1-11, wherein R1 is a phenyl optionally substituted with one R11, wherein said R11 is located in the meta position of said phenyl.
E20. The compound according to any of embodiments 1-11, wherein R1 is a phenyl optionally substituted with one R11, wherein said R11 is located in the ortho position of said phenyl.
E21. The compound according to any of embodiments 17-20, wherein said R11 is selected from methyl, trifluoromethyl, fluorine, hydroxy, methoxy, difluoromethoxy, trifluoromethoxy, cyano, methylsulfonyl, —S(O)₂NH₂ and —N(CH₃)₂.
E22. The compound according to any of embodiments 17-20, wherein said R11 is selected from C₁₋₆alkyl, halogen, hydroxy, haloC₁₋₆alkyl, C₁₋₆alkoxy and cyano.
E23. The compound according to embodiment 22, wherein said R11 represents C₁₋₆alkoxy.
E24. The compound according to embodiment 23, wherein said R11 represents methoxy.
E25. The compound according to embodiment 22, wherein said R11 represents C₁₋₆alkyl.
E26. The compound according to embodiment 25, wherein said R11 represents methyl.
E27. The compound according to any of embodiments 1-26, wherein R2, R3, R4, R5, R6 and R7 are selected independently of each other from H, C₁₋₆alkyl, C₂₋₆alkenyl, C₂₋₆alkynyl and C₁₋₆alkoxy.
E28. The compound according to embodiment 27, wherein R2, R3, R4, R5, R6 and R7 are selected independently of each other from H, methyl and methoxy.
E29. The compound according to any of embodiments 1-28, wherein R2 is methyl.
E30. The compound according to any of embodiments 1-29, wherein at least one of R3 and R7 is selected independently from methyl and methoxy.
E31. The compound according to embodiment 30, wherein both of R3 and R7 are selected independently of each other from methyl and methoxy.
E32. The compound according to any of embodiments 1-31, wherein R4. R5 and R6 are hydrogen.
E33. The compound according to any of embodiments 1-32, wherein R8 is selected from H, C₂₋₆alkenyl, C₂₋₆alkynyl and phenyl.
E34. The compound according to embodiment 33, wherein R8 is selected from H, methyl and phenyl.
E35. The compound according to embodiment 33, wherein R8 is C₁₋₆alkyl.
E36. The compound according to embodiment 35, wherein R8 is methyl.
E37. A compound according to embodiment 1 selected from
1: 1-(4-Methoxy-phenyl)-3,6-dimethyl-1H-pyrazolo[3,4-b]pyridine-4-carboxylic acid (2,4-dimethyl-pyridin-3-yl)-amide;
2: 3,6-Dimethyl-1-phenyl-1H-pyrazolo[3,4-b]pyridine-4-carboxylic acid (2,4-dimethyl-pyridin-3-yl)-amide;
3: 3,6-Dimethyl-1-phenyl-1H-pyrazolo[3,4-b]pyridine-4-carboxylic acid (3,5-dimethyl-pyridin-4-yl)-amide;
4: 1-(2-Methoxy-phenyl)-3,6-dimethyl-1H-pyrazolo[3,4-b]pyridine-4-carboxylic acid (2,4-dimethyl-pyridin-3-yl)-amide;
5: 1-(4-Methoxy-phenyl)-3,6-dimethyl-1H-pyrazolo[3,4-b]pyridine-4-carboxylic acid (2-methyl-pyridin-3-yl)-amide;
6: 3,6-Dimethyl-1-p-tolyl-1H-pyrazolo[3,4-b]pyridine-4-carboxylic acid (2,4-dimethyl-pyridin-3-yl)-amide;
7: 1-(4-Methoxy-phenyl)-3,6-dimethyl-1H-pyrazolo[3,4-b]pyridine-4-carboxylic acid (2-methoxy-4-methyl-pyridin-3-O-amide;
8: 1-(4-Methoxy-phenyl)-3,6-dimethyl-1H-pyrazolo[3,4-b]pyridine-4-carboxylic acid (4-methyl-pyridin-3-yl)-amide;
9: 1-(3-Methoxy-phenyl)-3,6-dimethyl-1H-pyrazolo[3,4-b]pyridine-4-carboxylic acid (2,4-dimethyl-pyridin-3-yl)-amide;
10: 3,6-Dimethyl-1-(6-methyl-pyridin-3-yl)-1H-pyrazolo[3,4-b]pyridine-4-carboxylic acid (2,4-dimethyl-pyridin-3-yl)-amide;
11: 1-(4-Methoxy-phenyl)-6-methyl-1H-pyrazolo[3,4-b]pyridine-4-carboxylic acid (2,4-dimethyl-pyridin-3-yl)-amide;
12: 1-(4-Methoxy-phenyl)-3,6-dimethyl-1H-pyrazolo[3,4-b]pyridine-4-carboxylic acid (4,6-dimethyl-pyrimidin-5-yl)-amide;
13: 1-(4-Fluoro-phenyl)-3,6-dimethyl-1H-pyrazolo[3,4-b]pyridine-4-carboxylic acid (2,4-dimethyl-pyridin-3-yl)-amide;
14: 1-(4-Cyano-phenyl)-3,6-dimethyl-1H-pyrazolo[3,4-b]pyridine-4-carboxylic acid (2,4-dimethyl-pyridin-3-yl)-amide;
15: 1-(4-Methoxy-phenyl)-3-methyl-1H-pyrazolo[3,4-b]pyridine-4-carboxylic acid (2,4-dimethyl-pyridin-3-yl)-amide;
16: 1-(6-Methoxy-pyridin-3-yl)-3,6-dimethyl-1H-pyrazolo[3,4-b]pyridine-4-carboxylic acid (2,4-dimethyl-pyridin-3-yl)-amide;
17: 3,6-Dimethyl-1-(4-trifluoromethoxy-phenyl)-1H-pyrazolo[3,4-b]pyridine-4-carboxylic acid (2,4-dimethyl-pyridin-3-yl)-amide;
18: 1-(4-Methoxy-phenyl)-3,6-dimethyl-1H-pyrazolo[3,4-b]pyridine-4-carboxylic acid (2,4,6-trimethyl-pyridin-3-yl)-amide;
19: 1-(4-Difluoromethoxy-phenyl)-3,6-dimethyl-1H-pyrazolo[3,4-b]pyridine-4-carboxylic acid (2,4-dimethyl-pyridin-3-yl)-amide;
20: 1-(4-Methoxy-phenyl)-3,6-dimethyl-1H-pyrazolo[3,4-b]pyridine-4-carboxylic acid (4-methyl-pyrimidin-5-yl)-amide;
21: 3,6-Dimethyl-1-(4-sulfamoyl-phenyl)-1H-pyrazolo[3,4-b]pyridine-4-carboxylic acid (2,4-dimethyl-pyridin-3-yl)-amide;
22: 3,6-Dimethyl-1-(4-trifluoromethyl-phenyl)-1H-pyrazolo[3,4-b]pyridine-4-carboxylic acid (2,4-dimethyl-pyridin-3-yl)-amide;
23: 1-(4-Dimethylamino-phenyl)-3,6-dimethyl-1H-pyrazolo[3,4-b]pyridine-4-carboxylic acid (2,4-dimethyl-pyridin-3-0)-amide;
24: 3-Methoxy-1-(4-methoxy-phenyl)-6-methyl-1H-pyrazolo[3,4-b]pyridine-4-carboxylic acid (2,4-dimethyl-pyridin-3-yl)-amide;
25: 3,6-Dimethyl-1-thiophen-3-yl-1H-pyrazolo[3,4-b]pyridine-4-carboxylic acid (2,4-dimethyl-pyridin-3-yl)-amide;
26: 3,6-Dimethyl-1-pyrimidin-5-yl-1H-pyrazolo[3,4-b]pyridine-4-carboxylic acid (2,4-dimethyl-pyridin-3-yl)-amide;
27: 3,6-Dimethyl-1-phenyl-1H-pyrazolo[3,4-b]pyridine-4-carboxylic acid (2-methoxy-4-methyl-pyridin-3-yl)-amide;
28: 3,6-Dimethyl-1-phenyl-1H-pyrazolo[3,4-b]pyridine-4-carboxylic acid (4-methoxy-2-methyl-pyridin-3-yl)-amide;
29: 3,6-Dimethyl-1-phenyl-1H-pyrazolo[3,4-b]pyridine-4-carboxylic acid (4-methoxy-6-methyl-pyridin-3-yl)-amide;
30: 1-(4-Methoxy-phenyl)-3,6-dimethyl-1H-pyrazolo[3,4-b]pyridine-4-carboxylic acid (4-dimethylamino-pyridin-3-yl)-amide;
31: 3,6-Dimethyl-1-phenyl-1H-pyrazolo[3,4-b]pyridine-4-carboxylic acid (2-methyl-pyridin-3-yl)-amide;
32: 3,6-Dimethyl-1-phenyl-1H-pyrazolo[3,4-b]pyridine-4-carboxylic acid (4-methyl-pyridin-3-yl)-amide;
33: 1-(4-Methoxy-phenyl)-3,6-dimethyl-1H-pyrazolo[3,4-b]pyridine-4-carboxylic acid (4,6-dimethyl-pyridin-3-yl)-amide;
34: 1-(4-Methanesulfonyl-phenyl)-3,6-dimethyl-1H-pyrazolo[3,4-b]pyridine-4-carboxylic acid (2,4-dimethyl-pyridin-3-yl)-amide;
35: 1-(4-Methoxy-phenyl)-3-methyl-6-phenyl-1H-pyrazolo[3,4-b]pyridine-4-carboxylic acid (2,4-dimethyl-pyridin-3-yl)-amide;
36: 1-(4-Methoxy-phenyl)-3,6-dimethyl-1H-pyrazolo[3,4-b]pyridine-4-carboxylic acid (4-cyano-pyridin-3-yl)-amide;
37: 1-(4-Hydroxy-phenyl)-3,6-dimethyl-1H-pyrazolo[3,4-b]pyridine-4-carboxylic acid (2,4-dimethyl-pyridin-3-yl)-amide;
   and pharmaceutically acceptable salts of any of these compounds.
   E38. A compound according to any of embodiments 1-37 for use as a medicament.
   E39. A compound according to any of embodiments 1-37, for use in therapy.
   E40. A compound according to any of embodiments 1-37, for use in the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes; weight control, inflammatory disorders, reduced angiogenesis; amyotrophic lateral sclerosis and pain.
   E41. The compound according to embodiment 40, wherein said a disease or disorder is selected from schizophrenia; AD; ADHD; autism spectrum disorders; PD; amyotrophic lateral sclerosis; Huntington's disease; dementia associated with Lewy bodies and pain.
   E42. The compound according to embodiment 41, wherein said disease or disorder is selected from schizophrenia; AD; ADHD and autism spectrum disorders.
   E43. The compound according to embodiment 42, wherein, said disease or disorder is selected from negative and/or cognitive symptoms of schizophrenia.
   E44. The compound according to any of embodiments 1-37, for use concomitantly or sequentially with a therapeutically effective amount of a compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers in the treatment of a disease or disorder according to any of embodiments 40-43.
   E45. A pharmaceutical composition comprising a compound according to any of embodiments 1-37, and one or more pharmaceutically acceptable carrier or excipient.
   E46. The composition according to embodiment 45, which composition additionally comprises a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E47. The composition according to embodiment 46; wherein said second compound is an acetylcholinesterase inhibitor.
   E48. A kit comprising a compound according to any of embodiments 1-37, together with a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E49. The kit according to embodiment 48, wherein said second compound is an acetylcholinesterase inhibitor.
   E50. A method for the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain, which method comprises the administration of a therapeutically effective amount of a compound according to any of embodiments 1-37 to a patient in need thereof.
   E51. The method according to embodiment 50, wherein said disease or disorder is selected from schizophrenia; AD; ADHD; autism spectrum disorders; PD; amyotrophic lateral sclerosis; Huntington's disease; dementia associated with Lewy bodies and pain.
   E52. The method according to embodiment 51, wherein said disease or disorder is selected from schizophrenia; AD; ADHD and autism spectrum disorders.
   E53. The method according to embodiment 52, wherein said treatment comprises the treatment of negative and/or cognitive symptoms of schizophrenia.
   E54. The method according to any of embodiments 50-53, wherein said treatment further comprises the administration of a therapeutically effective amount of a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E55. The method according to embodiment 54, wherein said second compound, is an acetylcholinesterase inhibitor.
   E56. Use of a compound according to any of embodiments 1-37, for the manufacture of a medicament for the treatment of a disease or disorder selected from Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory Impairment (AAMI); senile dementia; AIDS dementia; Pick's disease; dementia associated with Lewy bodies; dementia associated with Down's syndrome; Huntington's Disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.
   E57: The use according to embodiment 56, wherein said disease or disorder is Selected from schizophrenia; AD; ADHD; autism spectrum disorders; PD; amyotrophic lateral sclerosis; Huntington's disease; dementia associated with Lewy bodies and pain.
   E58. The use according to embodiment 57, wherein said disease or disorder is selected from schizophrenia; AD; ADHD and autism spectrum disorders.
   E59. The use according to embodiment 58, wherein said disease or disorder is selected from the positive, negative and/or cognitive symptoms of schizophrenia.
   E60. The use according to any of embodiments 56-59, wherein said medicament further comprises a second compound selected from the list consisting of acetylcholinesterase inhibitors; glutamate receptor antagonists; dopamine transport inhibitors; noradrenalin transport inhibitors; D2 antagonists; D2 partial agonists; PDE10 antagonists; 5-HT2A antagonists; 5-HT6 antagonists; KCNQ antagonists; lithium; sodium channel blockers and GABA signaling enhancers.
   E61. The use according to embodiment 60, wherein said second compound is an acetylcholinesterase inhibitor.

The compounds of the invention may exist in unsolvated as well as in solvated forms in which the solvent molecules are selected from pharmaceutically acceptable solvents such as water, ethanol and the like. In general, such solvated forms are considered equivalent to the unsolvated forms for the purposes of this invention.

The compounds of the present invention may have one or more asymmetric centres and it is intended that any optical isomers (i.e. enantiomers or diastereomers), in the form of separated, pure or partially purified optical isomers and any mixtures thereof including racemic mixtures, i.e. a mixture of stereoisomers; are included within the scope of the invention. In this context is understood that when specifying the enantiomeric form, then the compound is in enantiomeric excess, e.g. essentially in a pure, mono-enantiomeric form.

Racemic forms can be resolved into the optical antipodes by known methods, for example by separation of diastereomeric salts thereof with an optically active acid, and liberating the optically active amine compound by treatment with a base. Another method for resolving racemates into the optical antipodes is based upon chromatography of an optically active matrix. The compounds of the present invention may also be resolved by the formation of diastereomeric derivatives. Additional methods for the resolution of optical isomers, known to those skilled in the art, may be used. Such methods include those discussed by J. Jaques, A. Collet and S. Wilen in “Enantiomers, Racemates, and Resolutions”, John Wiley and Sons, New York (1981). Optically active compounds can also be prepared from optically active starting materials.

Furthermore, when a double bond or a fully or partially saturated ring system is present in the molecule geometric isomers may be formed. It is intended that any geometric isomers, as separated, pure or partially purified geometric isomers or mixtures thereof are included within the scope of the invention. Likewise, molecules having a bond with restricted rotation may form geometric isomers. These are also intended to be included within the scope of the present invention.

Furthermore, some of the compounds of the present invention may exist in different tautomeric forms and it is intended that any tautomeric forms that the compounds are able to form are included within the scope of the present invention.

The compounds of the present invention may be administered alone as a pure compound or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19 Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings.

Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include suppositories, sprays, ointments, cremes, gels, inhalants, dermal patches, implants, etc.

In one embodiment, the compound of the present invention is administered in an amount from about 0.001 mg/kg body weight to about 100 mg/kg body weight per day. In particular, daily dosages may be in the range of 0.01 mg/kg body weight to about 50 mg/kg body weight per day. The exact dosages will depend upon the frequency and mode of administration, the sex, the age the weight, and the general condition of the subject to be treated, the nature and the severity of the condition to be treated, any concomitant diseases to be treated, the desired effect of the treatment and other factors known to those skilled in the art.

A typical oral dosage for adults will be in the range of 0.1-1000 mg/day of a compound of the present invention, such as 1-500 mg/day, such as 1-100 mg/day or 1-50 mg/day. Conveniently, the compounds of the invention are administered in a unit dosage form containing said compounds in an amount of about 0.1 to 500 mg, such as 10 mg, 50 mg 100 mg, 150 mg, 200 mg or 250 mg of a compound of the present invention.

For parenteral administration, solutions of the compound of the invention in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospho lipids, fatty acids, fatty acid amines, polyoxyethylene and water. The pharmaceutical compositions formed by combining the compound of the invention and the pharmaceutical acceptable carriers are then readily administered, in a variety of dosage forms suitable for the disclosed routes of administration.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tablet, e.g. placed in a hard gelatine capsule in powder or pellet form or in the form of a troche or lozenge. The amount of solid carrier may vary but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

Tablets may be prepared by mixing the active ingredient with ordinary adjuvants and/or diluents followed by the compression of the mixture in a conventional tabletting machine. Examples of adjuvants or diluents comprise: Corn starch, potato starch, talcum, magnesium stearate, gelatine, lactose, gums, and the like. Any, other adjuvants or additives usually used for such purposes such as colourings, flavourings, preservatives etc. may be used provided that they are compatible with the active ingredients.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). It should be understood that the various aspects, embodiments, implementations and features of the invention mentioned herein may be claimed separately, or in any combination.

### General Synthetic Schemes

The compounds of formula [I] may be prepared by methods described below, together with synthetic methods known in the art of organic chemistry, or modifications that are familiar to those of ordinary skill in the art. The starting materials used herein are available commercially or may be prepared by routine methods known in the art, such as those method described in standard reference books such as “Compendium of Organic Synthetic Methods, Vol. I-XII” (published with Wiley-Interscience). Preferred methods include, but are not limited to, those described below.

The schemes are representative of methods useful in synthesizing the compounds of the present invention. They are not to constrain the scope of the invention in any way.

### Experimental Procedures and Working Examples

Synthesis of various compounds of the present invention are described in the following. Additional compounds within the scope of this invention may be prepared using the methods illustrated in these examples, either alone or in combination with techniques generally known in the art.

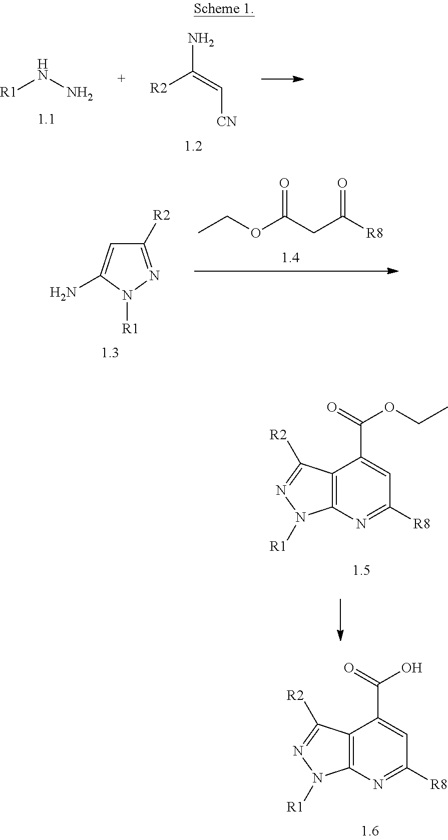

Scheme 1 illustrates a method for preparing compounds depicted by formula 1.6. This method involves the formation of a substituted pyrazole of formula 1.3. The synthesis of pyrazoles of formula 1.3 can as an example be achieved by heating enamines of formula 1.2 with hydrazines of formula 1.1 in a solvent such as ethanol, isopropanol, aqueous HCl solution, aqueous acetic acid with or without a base additive such as triethyl amine. A range of other methods have also been described in the literature to give compounds of formula 1.3 (for example Beilstein J. Org. Chem., 2011, 7, 179-197). Substituted pyrazoles of formula 1.3 can react with compounds of formula 1.4 to give compounds depicted by formula 1.5 by heating a mixture of the compounds in a solvent such as acetic acid. The ester functionality of compounds of formula 1.5 can be hydrolyzed to provide the corresponding acid derivative of formula 1.6 for example by treatment with aqueous base such as KOH or LiOH in a solvent such as MeOH or THF.

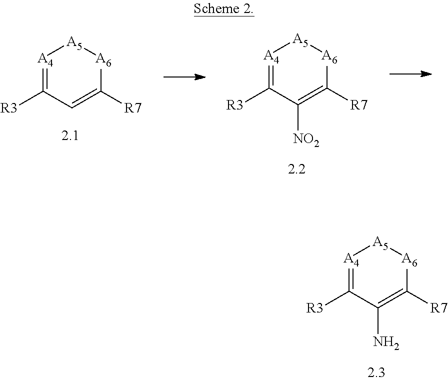

Scheme 2 illustrates a method for preparing compounds depicted by formula 2.3. Nitro compounds depicted by formula 2.2 can be prepared by nitrating compounds depicted by formula 2.1 by treatment under nitrating conditions such as aqueous HNO₃ in H₂SO₄, KNO₃ in H₂SO₄ or methods based on N₂O₅ (Synthesis, 1997, 281-283). Nitro compounds depicted by formula 2.2 can be reduced to compounds depicted by formula 2.3 by reductive methods, such as hydrogenation using Pd/C catalysts in solvents such as EtOH or THF, or treatment with Fe and a proton source such as NH₄Cl in solvents such as EtOH.

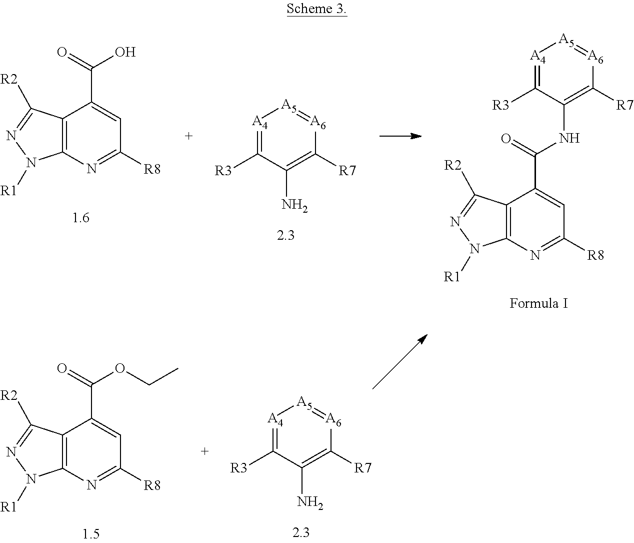

Scheme 3 illustrates methods for preparing compounds of formula I. The carboxylic acid of formula 1.6 can be coupled to amines of the formula 2.3 using EDC and HOBt or another suitable coupling reagent in the presence of a base such as diisopropyl ethylamine or N-methyl morpholine in a solvent such as DMF or DMAC. Other methods for preparing compounds of formula I includes treating a mixture of a carboxylic acid of formula 1.6 with an amine of the formula 2.3 with POCl₃ in warm pyridine or treating a preformed salt of carboxylic acid of the formula 1.6 and amine of the formula 2.3 with T3P.

Scheme 3 also illustrates a method for preparing compounds of the formula I in which carboxylic esters of formula 1.5, or the corresponding methyl ester, react with a preformed complex between an amine of the formula 2.3 and an alane reagent such as AlMe₃ in a solvent such as toluene or CH₂Cl₂.

### EXAMPLES

The invention will be illustrated by the following non-limiting examples.

### ABBREVIATIONS

br=broad. Brine=saturated aqueous solution of sodium chloride. Conc.=concentrated. d=doublet. DIPEA=N,N-Diisopropylethylamine. DMAC=N,N-Dimethylacetamide. DMAP=4-(dimethylamino)pyridine. DMF=dimethyl formamide. DMSO=dimethyl sulfoxide. EDC=1-Ethyl-3-(3-Dimethylaminopropyl)carbodiimide. EtOAc=ethyl acetate. h=hours. HATU=O-(7-Azabenzotriazol-1-yl)-N,N,N′,N′-tetramethyluronium hexafluorophosphate. HOBt=1-Hydroxybenzotriazole. HPLC=High Performance Liquid Chromatography. LC=Liquid Chromatography. m multiplet. min=minutes. mL=milliliters. s=singlet. t=triplet. T3P=1-propanephosphonic acid cyclic anhydride. TFA=trifluoroacetic acid. THF=tetrahydrofuran.

### Spectroscopic Methods

Analytical LCMS was performed using a Waters Acquity UPLC-MS consisting of Waters Aquity including column manager, binary solvent manager, sample organizer, PDA detector (operating at 254 nm), ELS detector and SQ-MS equipped with APPI-source operating in positive ion mode (ESI-source, APCI-source positive ion mode, negative ion mode).

LC-conditions: The column was a Acquity UPLC BEH C18 1.7 μm; 2.1×50 mm operating at 60° C. with 1.2 ml/min of a binary gradient consisting of water+0.05% TFA (A) and acetonitrile+5% water+0.035% TFA (B).

| Gradient: | Time, min. | % B |
|---|---|---|
| | 0.00 | 10.0 |
| | 1.00 | 100 |
| | 1.01 | 10.0 |
| | 1.15 | 10.0 |

Preparative HPLC was performed using a Waters delta prep HPLC equipped with a Waters 2545 Binary gradient pump and a Waters 2998 PDA detector. Column Sunfire (30×250 mm) 10 μm particle size. Solvent system A=0.01 M Ammonium acetate:water and B=acetonitrile. Isocratic A:B=35:65 with a flow of 30 mL/min.

¹H NMR spectra were recorded at 500.13 MHz on a Bruker Avance DRX-500 instrument at T=303.3 K or at 600 MHz on a Bruker Avance AV-III-600 instrument or at 400 MHz on a Varian 400MR instrument at T=298.15 K or at 400 MHz on a Varian vnmrs instrument.

### Preparation of Intermediates

### Synthesis of Anilines

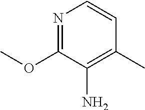

### IM01: 2-Methoxy-4-methyl-pyridin-3-ylamine

The title compound is commercially available. A mixture of 4-methyl-2-methoxy-3-nitropyridine (4.5 g, 26.8 mmol), NH₄Cl (7.16 g, 133.9 mmol) and Fe powder (7.5 g, 133.9 mmol) in ethanol (50 mL) was stirred at 25° C. The mixture was slowly heated to 90° C. and stirred at this temperature for 2 h. The reaction mixture was filtered through a plug of celite and the celite plug was washed with ethanol (50 mL). The combined filtrates were evaporated to dryness. The remanens was diluted with water (50 mL) and the mixture was extracted with EtOAc (100 mL). The organic layer was dried over Na₂SO₄. Evaporation to dryness gave the title compound as a solid (3.5 g, 81%). 1H NMR (CDCl₃, 400 MHz) δ 7.50-7.49 (1H, d, J=5.2 Hz), 6.62-6.61 (1H, d, J=5.2 Hz), 3.97 (3H, s), 3.79-3.69 (2H, br s), 2.15 (3H, s).

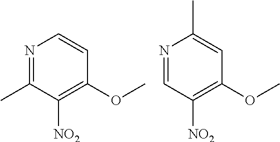

### IM02+IM03: Mixture of 4-Methoxy-2-methyl-3-nitro-pyridine and 4-Methoxy-2-methyl-5-nitro-pyridine, IM02+IM03

A mixture of (3.2 mL of conc H₂SO₄+3.2 mL of HNO₃) was slowly added drop wise to a solution of 4-methoxy-2-methylpyridine (4.8 g, 39.0 mmol) in H₂SO₄ (22.2 ml, conc) kept at 0° C. After completion of the addition the reaction temperature was slowly increased to 65. ° C. and the mixture was stirred at this temperature for 16 h. The reaction mixture was then poured into ice-water (50 ml) and filtered. The remanens was washed with water (50 mL) to give a mixture of the title compounds as a yellow solid (2.30 g, 35%). The mixture was used without further purification.

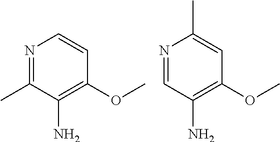

### IM04+IM05: Mixture of 4-Methoxy-2-methyl-pyridin-3-ylamine and 4-methoxy-6-methyl-pyridin-3-ylamine

To a solution of a mixture of compounds IM02 and IM03 (3.0 g, 17.9 mmol) in ethanol (50 mL) was added 10% Pd/C (300 mg). Then mixture was hydrogenated at 60 psi for 24 h at room temperature. The mixture was then filtered through a plug of celite. The celite was washed with ethanol (20 mL). The combined filtrates were evaporated to dryness to give a mixture of the title compounds as an off-white solid. (2.2 g, 89%). The mixture was used without further purification.

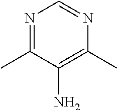

### IM06: 4,6-Dimethyl-pyrimidin-5-ylamine

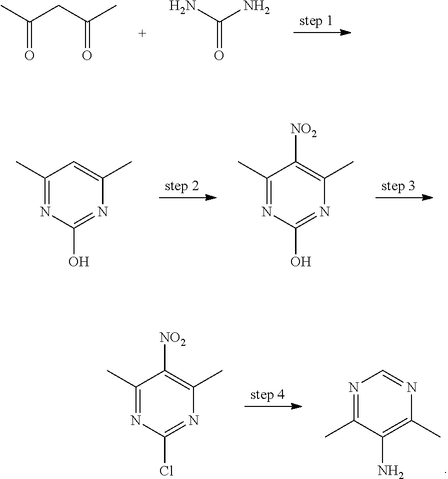

### Step 1:

To a solution of urea (20.0 g, 333.3 mmol) and acetyl acetone (33.3 g, 333.3 mmol) in EtOH (166 mL) was added concentrated aqueous HCl (45 mL). The mixture was stirred at 80° C. for 24 h. The mixture was then cooled to room temperature, filtered and the remanens washed with cold EtOH to give 4,6-dimethyl-pyrimidin-2-ol as a colorless solid (40 g, 97%). 1H NMR (DMSO-d6, 400 MHz) δ 7.35 (1H, s), 2.43 (6H, s).

### Step 2:

To a stirring solution of concentrated H₂SO₄ at 0° C. was added 4,6-dimethylpyrimidin-2-ol (10 g, 80.6 mmol). Fuming HNO₃ (7.8 mL) was then slowly added. Cooling was removed and the mixture was allowed to stir at room temperature for 5 h. The reaction mixture was poured into crushed ice and the pH was adjusted to approximately 4 by addition of aqueous NaOH. The mixture was extracted with EtOAc and the combined organic layers were evaporated to dryness to give crude 4,6-dimethyl-5-nitro-pyrimidin-2-ol which was used without further purification.

### Step 3:

4,6-Dimethyl-5-nitropyrimidin-2-ol 8 (1 g, 5.9 mmol) was dissolved in POCl₃ (10 mL) and the reaction mixture was heated to reflux for 5 h. The reaction mixture was cooled to room temperature and then poured in to ice-water. The mixture was extracted with EtOAc. The combined organic layers were washed with water and evaporated to dryness to give crude 2-chloro-4,6-dimethyl-5-nitro-pyrimidine (0.20 g, 18%) sufficiently pure for the next step.

### Step 4:

To a solution of 2-chloro-4,6-dimethyl-5-nitropyrimidine (50 mg, 0.26 mmol) in ethanol was added 10% Pd/C. The mixture was stirred under an atmosphere of H₂ at room temperature for 5 h. The reaction mixture was filtered through a pad of celite and concentrated in vacuo to give the title compound IM06 (20 mg, 63%). 1H NMR (DMSO-d6, 400 MHz) δ 7.17 (1H, s), 4.99 (2H, s), 2.26 (6H, s).

### Synthesis of pyrazol-3-ylamines

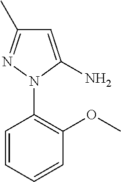

### IM07: 2-(2-Methoxy-phenyl)-5-methyl-2H-pyrazol-3-ylamine

To a solution of (2-methoxy-phenyl)-hydrazine HCl salt (10.0 g, 57.3 mmol) in concentrated aqueous HCl (50 mL) was added 3-amino-but-2-enenitrile (5.0 g, 60.9 mmol) at room temperature. The reaction temperature was increased to 100° C. and the mixture was stirred at this temperature for 18 h. The reaction mixture was cooled to room temperature and then poured into ice-cold 2N NaOH solution (200 mL). The resulting mixture was filtered. The remanens was washed with water (200 mL) and dried in vacuo to give the title compound as an off-white solid (10.0 g, 86%). 1H NMR (DMSO-d6, 400 MHz) δ 7.39-7.35 (1H, m), 7.24-7.22 (1H, m), 7.17-7.15 (1H, m), 7.03-6.99 (1H, m), 5.21 (1H, s), 4.76 (2H, s), 3.78 (3H, s), 2.02 (3H, s).

The following compounds were prepared analogously:

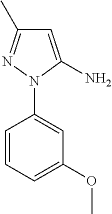

### IM08: 2-(3-Methoxy-phenyl)-5-methyl-2H-pyrazol-3-ylamine

The title compound was obtained as an off-white solid (1.5°g, 65%) sufficiently pure for the next step.

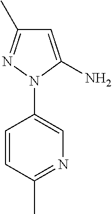

### IM09: 5-Methyl-2-(6-methyl-pyridin-3-yl)-2H-pyrazol-3-ylamine

The title compound was obtained as a yellow solid (1.6 g, 36%) sufficiently pure for the next step.

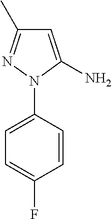

### IM10: 2-(4-Fluoro-phenyl)-5-methyl-2H-pyrazol-3-ylamine

The title compound was obtained as an off-white solid (4.0 g, 72%) sufficiently pure for the next step.

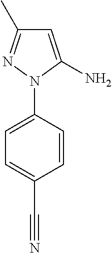

### IM11: 4-(5-Amino-3-methyl-pyrazol-1-yl)-benzonitrile

The title compound was obtained as an off-white solid (4.5 g, 77%) sufficiently pure for the next step.

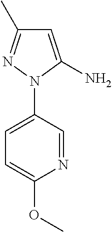

### IM12: 2-(6-Methoxy-pyridin-3-yl)-5-methyl-2H-pyrazol-3-ylamine

(6-Methoxy-pyridin-3-yl)-hydrazine was prepared from 6-methoxy-pyridin-3-ylamine as described in Org. Lett., 2009, 11, 22, 5142-5145. The title compound was obtained as an off-white solid (4.0 g, 48%) sufficiently pure for the next step.

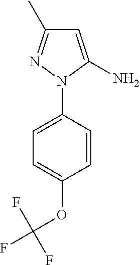 [SMILES: Cc1cc(N)n(-c2ccc(OC(F)(F)F)cc2)n1]

### IM13: 5-Methyl-2-(4-trifluoromethoxy-phenyl)-2H-pyrazol-3-ylamine

The title compound was obtained as an off-white solid (3.0 g, 72%) sufficiently pure for the next step.

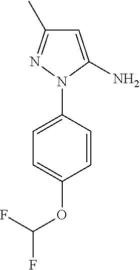 [SMILES: Cc1cc(N)n(-c2ccc(OC(F)F)cc2)n1]

### IM14: 2-(4-Difluoromethoxy-phenyl)-5-methyl-2H-pyrazol-3-ylamine

The intermediate (4-difluoromethoxy-phenyl)-hydrazine was prepared from 4-difluoromethoxy-phenylamine as described in Org. Lett, 2009, 11, 22, 5142-5145. The title compound was obtained as an off-white solid (4.0 g, 58%) sufficiently pure for the next step.

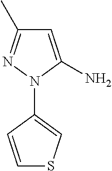

### IM15: 5-Methyl-2-thiophen-3-yl-2H-pyrazol-3-ylamine

The intermediate thiophen-3-yl-hydrazine was prepared as described in Organic Letters 2003, 4129-4131, followed by Boc deprotection using HCl in dioxane. The title compound was obtained as a yellow solid (1.5 g, 40%) after flash chromatography (silica, methylene chloride:MeOH=10:1).

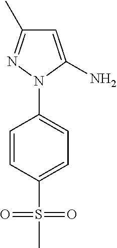 [SMILES: Cc1cc(N)n(-c2ccc(S(C)(=O)=O)cc2)n1]

### IM16: 2-(4-Methanesulfonyl-phenyl)-5-methyl-2H-pyrazol-3-ylamine

To a solution of (4-methanesulfonyl-phenyl)-hydrazine (1.5 g, 6.76 mmol) in EtOH (20 mL) was added 3-amino-but-2-enenitrile (0.55 g, 6.76 mmol). The resulting mixture was refluxed for 8 h. The mixture was concentrated in vacuo and the residue was extracted with EtOAc (2×200 mL). The combined organic layers were washed with saturated aqueous NaHCO₃ solution (100 ml), dried over Na₂SO₄ and evaporated to dryness. Flash chromatography (silica, methylene chloride:MeOH=10:1) gave compound IM16 as a solid (1.7 g, 99%).

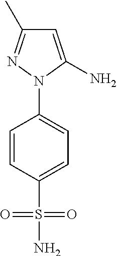 [SMILES: Cc1cc(N)n(-c2ccc(S(N)(=O)=O)cc2)n1]

### IM17: 4-(5-Amino-3-methyl-pyrazol-1-yl)-benzenesulfonamide

The title compound was obtained as an off-white solid (2.31 g, 86%) sufficiently pure for the next step.

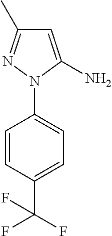 [SMILES: Cc1cc(N)n(-c2ccc(C(F)(F)F)cc2)n1]

### IM18: 5-Methyl-2-(4-trifluoromethyl-phenyl)-2H-pyrazol-3-ylamine

The title compound was obtained as a solid (4.1 g, 71%) sufficiently pure for the next step.

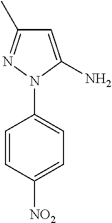 [SMILES: Cc1cc(N)n(-c2ccc([N+](=O)[O-])cc2)n1]

IM19: 5-Methyl-2-(4-nitro-phenyl)-2H-pyrazol-3-ylamine

The title compound was obtained as a solid (7.1 g, 99%).

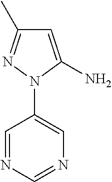

### IM20: 5-Methyl-2-pyrimidin-5-yl-2H-pyrazol-3-ylamine

The intermediate pyrimidin-5-yl-hydrazine was prepared as described in Tetrahedron Lett., 51 (2010), 5005-5008, followed by Boc deprotection using HCl in dioxane. The title compound was obtained as a solid (3.85 g, 99%).

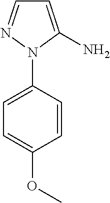

### IM21: 2-(4-Methoxy-phenyl)-2H-pyrazol-3-ylamine

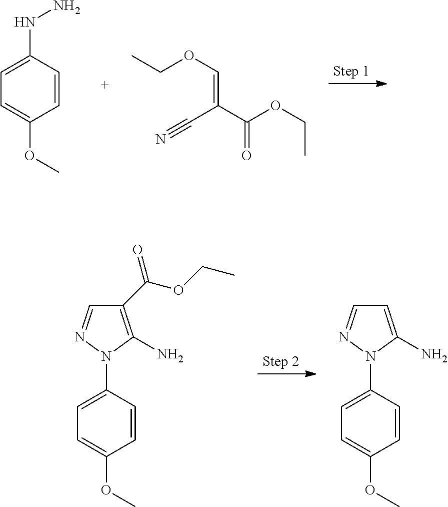

### Step 1:

To a solution of 4-methoxyphenyl hydrazine 1 (500 mg, 3.59 mmol) in ethanol (3 mL) was added ethyl 2-cyano-3-ethoxyacrylate 2 (600 mg, 3.53 mmol) at room temperature. The reaction temperature was increased to 85° C. and the mixture was stirred at this temperature for 10 h. The reaction mixture was cooled to room temperature and then poured into ice-cold 2N NaOH solution (10 mL). This mixture was filtered and the remanens dried in vacuo to give the crude ethyl ester (0.65 g, 70%). 1H NMR (DMSO-d6, 400 MHz) δ 7.65 (1H, s), 7.43-7.40 (2H, d), 7.08-7.06 (2H, d), 6.17 (2H, s), 4.23-4.18 (2H, m), 3.81 (3H, s), 1.28-1.24 (3H, t).

### Step 2:

To a solution of 5-amino-1-(4-methoxy-phenyl)-1H-pyrazole-4-carboxylic acid ethyl ester (8.9 g, 34.1 mmol) in concentrated aqueous HCl (100 mL) was stirred at 100° C. for 10 h. The reaction mixture was cooled to room temperature and then poured into ice-cold 1N NaOH solution (100 mL). The mixture was extracted with methylene chloride (2×150 mL). The combined organic layers were washed with water, dried over Na₂SO₄ and concentrated in vacuo to give title compound IM21 as an off-white solid (3.0 g, 16%). 1H NMR (DMSO-d6, 400 MHz) δ 7.46-7.42 (2H, m), 7.23-7.22 (1H, d), 7.04-7.0 (2H, m), 5.44-5.43 (1H, d), 5.16 (2H, s), 3.78 (3H, s).

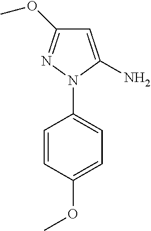 [SMILES: COc1ccc(-n2nc(OC)cc2N)cc1]

### IM22: 5-Methoxy-2-(4-methoxy-phenyl)-2H-pyrazol-3-ylamine

To a solution (4-methoxy-phenyl)-hydrazine (1.7 g, 0.01 mol) in MeOH (20 mL) was added 3,3-dimethoxy-acrylonitrile (1.1 g, 10 mmol). The resulting mixture was heated to reflux overnight. The mixture was concentrated in vacuo and the residue was extracted with EtOAc (200 mL). The organic layer was washed with saturated aqueous NaHCO₃ solution (100 ml), dried over Na₂SO₄ and evaporated to dryness. Flash chromatography (silica, methylene chloride:MeOH=10:1) gave the title compound IM22 as a solid (1.2 g, 57%). 1H NMR (DMSO-d6 400 MHz) δ 7.41 (d, 2H), 6.99 (d, 2H), 5.25 (s, 2H), 4.94 (s, 1H), 3.78 (s, 3H), 3.72 (s, 3H).

### Synthesis of pyrazolo[3,4-b]pyridine-4-carboxylic acid ethyl esters

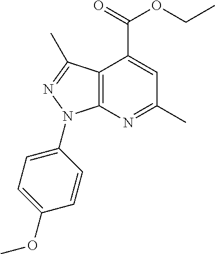 [SMILES: CCOC(=O)c1cc(C)nc2c1c(C)nn2-c1ccc(OC)cc1]

### IM23: 1-(4-Methoxy-phenyl)-3,6-dimethyl-1H-pyrazolo[3,4-b]pyridine-4-carboxylic acid ethyl ester

A round bottomed flask was charged with 2-(4-methoxy-phenyl)-5-methyl-2H-pyrazol-3-ylamine (2.0 g, 9.85 mmol) in acetic acid (10 mL). To this mixture was added ethyl-2,4-dioxovalerate (1.55 g, 9.85 mmol) and the mixture was subsequently stirred at 125° C. for 16 h. The mixture was cooled to room temperature and poured into ice-cold 2N NaOH solution (50 mL). The mixture was filtered and the remanens was washed with water (50 mL) and dried. Purification of the crude material by flash chromatography (silica, 10%. EtOAc in heptanes) gave the title compound as a solid (1.7 g, 53%). 1H NMR (400 MHz, CDCl₃) δ 8.09-8.02 (m, 2H), 7.47 (1H, s), 7.04-7.02 (m, 2H), 4.52-4.47 (m, 2H), 3.86 (3H, s), 2.75 (3H, s), 2.72 (3H, s), 1.49-1.45 (3H, t, J=7.2 Hz).

The following compounds were prepared analogously:

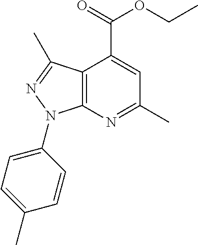 [SMILES: CCOC(=O)c1cc(C)nc2c1c(C)nn2-c1ccc(C)cc1]

### IM24: 3,6-Dimethyl-1-p-tolyl-4H-pyrazolo[3,4-b]pyridine-4-carboxylic acid ethyl ester

The crude compound was purified by flash chromatography (silica, 10% EtOAc in hexanes) to give the title compound as an off-white solid (1.4 g, 85%).

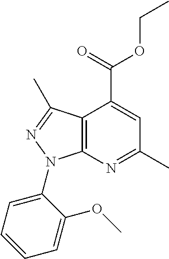 [SMILES: CCOC(=O)c1cc(C)nc2c1c(C)nn2-c1ccccc1OC]

### IM25: 1-(2-Methoxy-phenyl)-3,6-dimethyl-1H-pyrazolo[3,4-b]pyridine-4-carboxylic acid ethyl ester

Prepared using IM07. The crude compound was purified by flash chromatography (silica, 10% EtOAc in hexanes) to give the title compound as an off-white solid (1.2 g, 38%). 1H NMR (400 MHz, CDCl₃) δ 7.46-7A1 (3H, m), 7.11-7.07 (2H, m), 4.53-4.47 (2H, m), 3.76 (3H, s), 2.75 (3H, s), 2.64 (3H, s), 1.49-1.45 (3H, t, J=7.6 Hz).

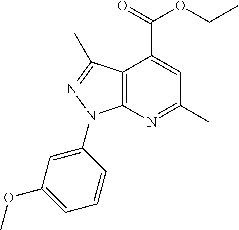 [SMILES: CCOC(=O)c1cc(C)nc2c1c(C)nn2-c1cccc(OC)c1]

### IM26: 1-(3-Methoxy-phenyl)-3,6-dimethyl-1H-pyrazolo[3,4-b]pyridine-4-carboxylic acid ethyl ester

Prepared using IM08. The crude compound was purified by flash chromatography (silica, 10% EtOAc in hexanes) to give the title compound as a pale yellow solid (1.6 g, 66%). ¹H NMR (CDCl₃, 400 MHz) δ 7.91-7.87 (2H, m), 7.48 (1H, s), 7.41-7.37 (1H, m), 6.85-6.82 (1H, m) 4.52-4.47 (2H, m), 3.90 (3H, s), 2.75 (3H, s), 2.73 (3H, s) 1.49-1.45 (3H, t).

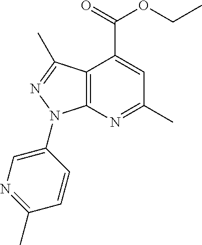 [SMILES: CCOC(=O)c1cc(C)nc2c1c(C)nn2-c1ccc(C)nc1]

### IM27: 3,6-Dimethyl-1-(6-methyl-pyridin-3-yl)-1H-pyrazolo[3,4-b]pyridine-4-carboxylic acid ethyl ester

Prepared using IM09. The crude compound was purified by flash chromatography (silica, 30% EtOAc in hexanes) to give the title compound as a pale yellow solid (1.5 g, 58%). ¹H NMR (CDCl3, 400 MHz) δ 9.46-9.45 (1H, d), 8.44-8.42 (1H, m), 7.51 (1H, s), 7.30-7.27 (1H, m), 4.52-4.47 (2H, m), 2.76 (3H, s), 2.72 (3H, s), 2.61 (3H, s), 1.49-1.45 (3H, t).

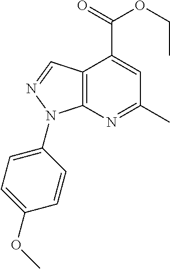 [SMILES: CCOC(=O)c1cc(C)nc2c1cnn2-c1ccc(OC)cc1]

### IM28: 1-(4-Methoxy-phenyl)-6-methyl-1H-pyrazolo[3,4-b]pyridine-4-carboxylic acid ethyl ester

Prepared using IM21. The crude compound was purified by flash chromatography (silica, 10% EtOAc in hexanes) to give the title compound as an off-white solid (3.4 g, 69%). ¹H NMR (CDCl3, 400 MHz) δ 8.50 (1H, s), 8.13-8.09 (2H, m), 7.67 (1H, s), 7.08-7.04 (2H, m), 4.55-4.49 (2H, m), 3.87 (3H, s), 2.76 (3H, s), 1.55-1.48 (3H, m).

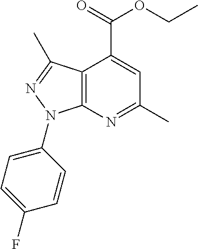 [SMILES: CCOC(=O)c1cc(C)nc2c1c(C)nn2-c1ccc(F)cc1]

### IM29: 1-(4-Fluoro-phenyl)-3,6-dimethyl-1H-pyrazolo[3,4-b]pyridine-4-carboxylic acid ethyl ester

Prepared using IM10. The crude compound was washed with, water to give the title compound as an off-white solid (1.6 g, 66%). 1H NMR (DMSO-d6, 300 MHz) δ 8.23-8:19 (2H, m), 7.57 (1H, s), 7.43-7.37 (2H, m), 4.49-4.42 (2H, m) 2.69 (3H, s), 2.65 (3H, s), 1.42-1.37 (3H, t).

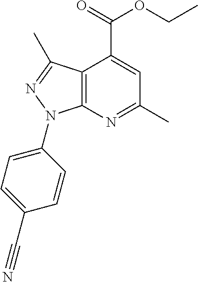 [SMILES: CCOC(=O)c1cc(C)nc2c1c(C)nn2-c1ccc(C#N)cc1]

### IM30: 1-(4-Cyano-phenyl)-3,6-dimethyl-1H-pyrazolo[3,4-b]pyridine-4-carboxylic acid ethyl ester

Prepared using IM11. The crude compound was washed with water to give the title compound as a pale, yellow solid (4.0 g, 83%). 1H NMR (CDCl₃, 400 MHz) δ 8.63-8.61 (2H, d), 7.79-7.77 (2H, d), 7.54 (1H, s) 4.53-4.47 (2H, m), 2.77 (3H, s), 2.75 (3H, s), 1.55-1.48 (3H, t).

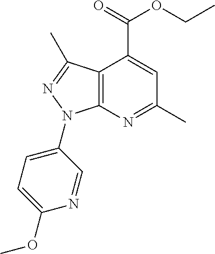 [SMILES: CCOC(=O)c1cc(C)nc2c1c(C)nn2-c1ccc(OC)nc1]

### IM31: 1-(6-Methoxy-pyridin-3-yl)-3,6-dimethyl-1H-pyrazolo[3,4-b]pyridine-4-carboxylic acid ethyl ester

Prepared using IM12. The crude compound was washed with water to give the title compound as a pale, yellow solid (4.0 g, 62%). ¹H-NMR (DMSO-d6, 400 MHz) δ 8.89-8.90 (1H, m), 8.37-8.34 (1H, m), 7.56 (1H, s), 7.04 (1H, s), 7.01 (1H, s), 4.48-4.42 (2H, m), 3.92 (3H, s), 2.68 (3H, s), 2.64 (3H, s), 1.41-1.38 (3H, m).

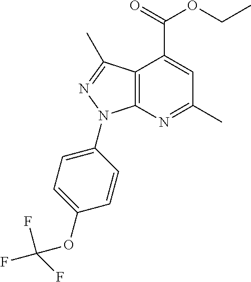 [SMILES: CCOC(=O)c1cc(C)nc2c1c(C)nn2-c1ccc(OC(F)(F)F)cc1]

### IM32: 3,6-Dimethyl-1-(4-trifluoromethoxy-phenyl)-1H-pyrazolo[3,4-b]pyridine-4-carboxylic acid ethyl ester

Prepared using IM13. The crude compound was washed with water to give the title compound as a pale, yellow solid (4.0 g, 66%). 1H NMR (DMSO-d6, 300 MHz) δ 8.36-8.34 (2H, m), 7.57-7.55 (2H, m), 7.56 (1H, s) 4.47-4.42 (2H, m), 2.69 (3H, s), 2.64 (3H, s), 1.41-1.38 (3H, m).

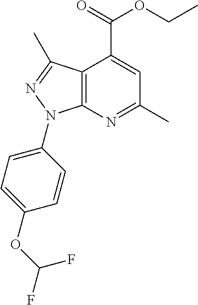 [SMILES: CCOC(=O)c1cc(C)nc2c1c(C)nn2-c1ccc(OC(F)F)cc1]

### IM33: 1-(4-Difluoromethoxy-phenyl)-3,6-dimethyl-1H-pyrazolo[3,4-b]pyridine-4-carboxylic acid ethyl ester

Prepared using IM14. The crude compound was washed with water to give the title compound as a pale, yellow solid (1.6 g, 26%). 1H NMR (DMSO-d6, 300 MHz) δ 8.25-8.23 (2H, m), 7.58 (1H, s), 7.47-7.10 (3H, m) 4.48-4.43 (2H, m), 2.70 (3H, s), 2.66 (3H, s), 1.42-1.37 (3H, m).

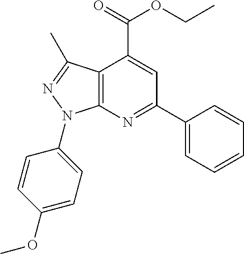 [SMILES: CCOC(=O)c1cc(-c2ccccc2)nc2c1c(C)nn2-c1ccc(OC)cc1]

### IM34: 1-(4-Methoxy-phenyl)-3-methyl-6-phenyl-1H-pyrazolo[3,4-b]pyridine-4-carboxylic acid ethyl ester

Prepared using 2-(4-methoxy-phenyl)-5-methyl-2H-pyrazol-3-ylamine and 2,4-dioxo-4-phenyl-butyric acid ethyl ester to give the title compound as a solid (2.5 g, 87%) sufficiently pure for the next step.

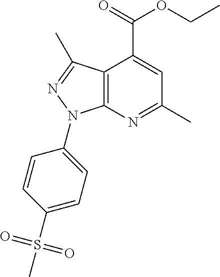 [SMILES: CCOC(=O)c1cc(C)nc2c1c(C)nn2-c1ccc(S(C)(=O)=O)cc1]

### IM35: 1-(4-Methanesulfonyl-phenyl)-3,6-dimethyl-1H-pyrazolo[3,4-b]pyridine-4-carboxylic acid ethyl ester

Prepared using IM16 to give the title compound as a solid (2.0 g, 79%) sufficiently pure for the next step.

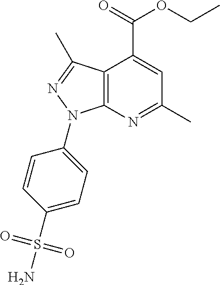 [SMILES: CCOC(=O)c1cc(C)nc2c1c(C)nn2-c1ccc(S(N)(=O)=O)cc1]

### IM36: 3,6-Dimethyl-1-(4-sulfamoyl-phenyl)-1H-pyrazolo[3,4-b]pyridine-4-carboxylic acid ethyl ester

Prepared using IM17 to give the title compound as a solid (1.97 g, 76%) sufficiently pure for the next step.

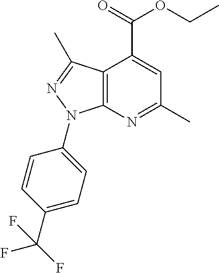 [SMILES: CCOC(=O)c1cc(C)nc2c1c(C)nn2-c1ccc(C(F)(F)F)cc1]

### IM37: 3,6-Dimethyl-1-(4-trifluoromethyl-phenyl)-1H-pyrazolo[3,4-b]pyridine-4-carboxylic acid ethyl ester

Prepared using IM18 to give the title compound as a solid (2.66 g, 58%) sufficiently pure for the next step.

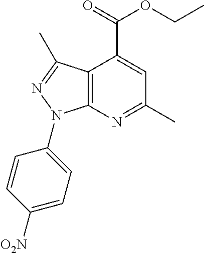 [SMILES: CCOC(=O)c1cc(C)nc2c1c(C)nn2-c1ccc([N+](=O)[O-])cc1]

### IM38: 3,6-Dimethyl-1-(4-nitro-phenyl)-1H-pyrazolo[3,4-b]pyridine-4-carboxylic acid ethyl ester

Prepared using IM19 to give the title compound as a solid (3.39 g, 31%) sufficiently pure for the next step.

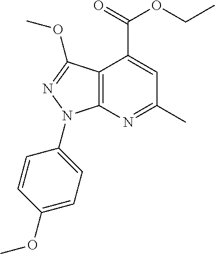 [SMILES: CCOC(=O)c1cc(C)nc2c1c(OC)nn2-c1ccc(OC)cc1]

### IM39: 3-Methoxy-1-(4-methoxy-phenyl)-6-methyl-1H-pyrazolo[3,4-b]pyridine-4-carboxylic acid ethyl ester

Prepared using IM22 to give the title compound as a solid (0.9 g, 48%). 1H NMR (CDCl₃ 400) δ 8.08 (d, 2H), 7.36 (s, 1H), 7.00 (d, 2H), 4.47 (q, 2H), 4.14 (s, 3H), 3.85 (s, 3H), 2.69 (s, 3H), 1.44 (t, 3H).

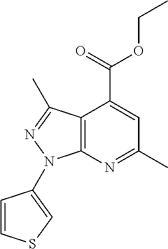 [SMILES: CCOC(=O)c1cc(C)nc2c1c(C)nn2-c1ccsc1]

### IM40: 3,6-Dimethyl-1-thiophen-3-yl-1H-pyrazolo[3,4-b]pyridine-4-carboxylic acid ethyl ester

Prepared using IM15 to give the title compound as a yellow solid (1.62 g, 64%). 1H NMR (CDCl3 400) δ 8.03-8.04 (m, 1H), 7.93-7.95 (m, 1H), 7.37-7.39 (m, 1H), 4.49 (m, 2H), 2.74 (s, 3H), 2.73 (s, 3H), 1.46 (t, J=7.2 Hz, 3H).

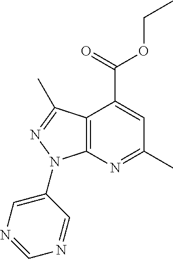 [SMILES: CCOC(=O)c1cc(C)nc2c1c(C)nn2-c1cncnc1]

### IM41: 3,6-Dimethyl-1-pyrimidin-5-yl-1H-pyrazolo[3,4-b]pyridine-4-carboxylic acid ethyl ester

Prepared using IM20 to give the title compound as a colorless solid (0.25 g, 4%). 1H NMR (DMSO-d6) δ 9.62 (s, 2H), 9.12 (s, 1H), 7.63 (s, 1H), 4.44 (m, 2H), 2.71 (s, 3H), 2.66 (s, 3 H), 1.37 (t, 3H).

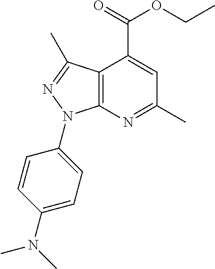 [SMILES: CCOC(=O)c1cc(C)nc2c1c(C)nn2-c1ccc(N(C)C)cc1]

### IM42: 1-(4-Dimethylamino-phenyl)-3,6-dimethyl-1H-pyrazolo[3,4-b]pyridine-4-carboxylic acid ethyl ester

To a solution of compound IM38 (1.63 g, 4.79 mmol) in THF (30 mL) and 5 mL of HCHO was added 5% Pd/C (2 g). The mixture was kept under H₂ (50 psi) for 12 h at room temperature. The mixture was filtered and the filtrate evaporated to dryness. Flash chromatography (silica, petroleum ether:EtOAc=20:1) gave the title compound as a yellow solid (0.62 g, 61%). ¹H NMR (CDCl₃, 400 MHz) δ 7.91 (d2H), 7.45 (s, 1H), 6.86 (d, 2H), 4.50 (m, 2H), 2.99 (s, 6 H), 2.78 (s, 3H), 2.74 (s, 3H), 1.47 (m, 3H).

### Synthesis of pyrazolo[3,4-b]pyridine-4-carboxylic acids

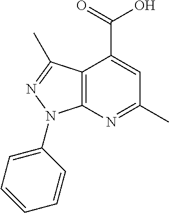 [SMILES: Cc1cc(C(=O)O)c2c(C)nn(-c3ccccc3)c2n1]

### IM43: 3,6-Dimethyl-1-phenyl-1H-pyrazolo[3,4-b]pyridine-4-carboxylic acid

A round bottomed flask was charged with 3,6-dimethyl-1-phenyl-1H-pyrazolo[3,4-b]pyridine-4-carboxylic acid ethyl ester (3.00 g, 10.2 mmol) in a mixture of THF (18 mL) and MeOH (18 mL). To this solution was added 2 M of Sodium hydroxide in Water (10.2 mL). The mixture was stirred at 80° C. for 1.5 hours and then evaporated to dryness. The resulting mixture was dissolved in a small amount of water and 2N HCl (aq.) was added until pH>3. The mixture was extracted with EtOAc. The combined organic layers wire dried over brine, Na₂SO₄ and evaporated to dryness to give the title compound as colorless crystals (2.51 g, 92%). 1H NMR (500 MHz, DMSO) δ 8.26-8.20 (m, 1H), 7.59-7.53 (m, 1H), 7.36-7.31 (m, 1H), 2.70 (s, 1H), 2.67 (s, 1H).

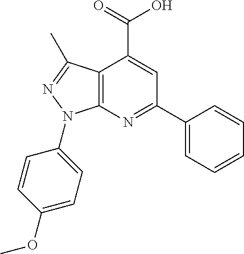 [SMILES: COc1ccc(-n2nc(C)c3c(C(=O)O)cc(-c4ccccc4)nc32)cc1]

### IM44: 1-(4-Methoxy-phenyl)-3-methyl-6-phenyl-1H-pyrazolo[3,4-b]pyridine-4-carboxylic acid

Prepared using IM34 to give the title compound as a yellow solid (2.26 g, 97%) sufficiently pure for the next step.

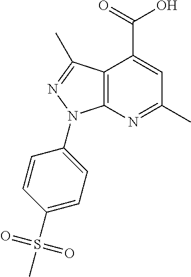 [SMILES: Cc1cc(C(=O)O)c2c(C)nn(-c3ccc(S(C)(=O)=O)cc3)c2n1]

### IM45: 1-(4-Methanesulfonyl-phenyl)-3,6-dimethyl-1H-pyrazolo[3,4-b]pyridine-4-carboxylic acid

Prepared using IM35 to give the title compound as a solid (1.61 g, 87%) sufficiently pure for the next step.

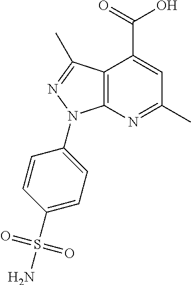 [SMILES: Cc1cc(C(=O)O)c2c(C)nn(-c3ccc(S(N)(=O)=O)cc3)c2n1]

### IM46: 3,6-Dimethyl-1-(4-sulfamoyl-phenyl)-1H-pyrazolo[3,4-b]pyridine-4-carboxylic acid

Prepared using IM36 to give the title compound as a solid (2.4 g, 99%) sufficiently pure for the next step. The hydrolysis was performed at room temperature.

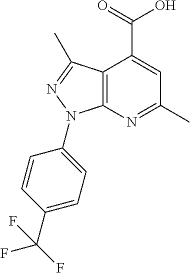 [SMILES: Cc1cc(C(=O)O)c2c(C)nn(-c3ccc(C(F)(F)F)cc3)c2n1]

### IM47: 3,6-Dimethyl-1-(4-trifluoromethyl-phenyl)-1H-pyrazolo[3,4-b]pyridine-4-carboxylic acid

Prepared using IM37 to give the title compound as a solid (1.6 g, 85%) sufficiently pure for the next step. The hydrolysis was performed at room temperature.

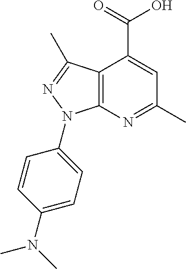

### IM48: 1-(4-Dimethylamino-phenyl)-3,6-dimethyl-1H-pyrazolo[3,4-b]pyridine-4-carboxylic acid

Prepared using IM42 to give the title compound as a solid (0.223 g, 97%) sufficiently pure for the next step.

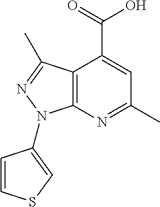 [SMILES: Cc1cc(C(=O)O)c2c(C)nn(-c3ccsc3)c2n1]

### IM49: 3,6-Dimethyl-1-thiophen-3-yl-1H-pyrazolo[3,4-b]pyridine-4-carboxylic acid

Prepared using IM40 to give the title compound as a yellow solid (0.41 g, 91%) sufficiently pure for the next step.

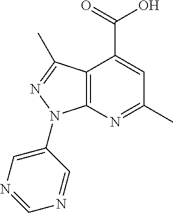 [SMILES: Cc1cc(C(=O)O)c2c(C)nn(-c3cncnc3)c2n1]

### IM50: 3,6-Dimethyl-1-pyrimidin-5-yl-1H-pyrazolo[3,4-b]pyridine-4-carboxylic acid

Prepared using IM41 to give the title compound as a solid (0.113 g, 62%) sufficiently pure for the next step.

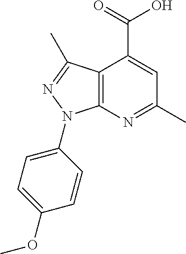

### IM51: 1-(4-Methoxy-phenyl)-3,6-dimethyl-1H-pyrazolo[3,4-b]pyridine-4-carboxylic acid

A round bottomed flask was charged with 1-(4-methoxy-phenyl)-3,6-dimethyl-1H-pyrazolo[3,4-b]pyridine-4-carboxylic acid ethyl ester IM23 (16.0 g, 49.2 mmol) in methanol/water (1:1, 600 mL). To this mixture was added LiOH hydrate (6.2 g, 147.6 mmol) at room temperature. The resulting reaction mixture was stirred for 16 h. Methanol was removed under reduced pressure and the resulting mixture diluted with water (500 mL). Acetic-acid was added to adjust pH>4. After stirring for 30 min the mixture was filtered and the remanens was washed with water and dried under vacuum. The crude material was washed with diethyl ether to give the title compound as a solid (13.5 g, 92%). 1H NMR (400 MHz, DMSO-d6) 13.89 (1H, br), 8.03-8.01 (m, 2H), 7.35 (1H, s), 7.12-7.10 (m, 2H, d), 3.82 (3H′, s), 2.66 (3H, s), 2.65 (3H, s).

The following compounds were prepared analogously:

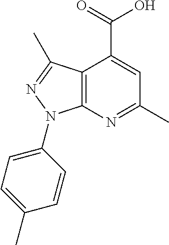 [SMILES: Cc1ccc(-n2nc(C)c3c(C(=O)O)cc(C)nc32)cc1]

### IM52: 3,6-Dimethyl-1-p-tolyl-1H-pyrazolo[3,4-b]pyridine-4-carboxylic acid

The crude compound was prepared from IM24 and subsequently washed with diethyl ether to give the title compound as a pale yellow powder (1.0 g, 79%). 1H NMR (DMSO-d6, 400 MHz) δ 13.89 (1H, br), 8.07-8.05 (2H, d), 7.52 (1H, s), 7.36-7.34 (2H, d), 2.68 (3H, s), 2.65 (3H, s), 2.37 (3H, s).

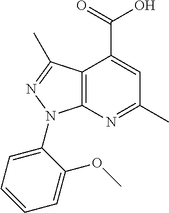 [SMILES: COc1ccccc1-n1nc(C)c2c(C(=O)O)cc(C)nc21]

### IM53: 1-(2-Methoxy-phenyl)-3,6-dimethyl-1H-pyrazolo[3,4-b]pyridine-4-carboxylic acid

The crude compound was prepared from IM25 and subsequently washed with diethyl ether to give the title compound as a pale yellow powder (0.9 g, 83%). 1H NMR (DMSO-d6, 400 MHz) δ 13.80 (1H, br), 7.54-7.5 (1H, m), 7.45 (1H, s), 7.39-7.37 (1H, m), 7.27-7.25 (1H, d), 7.12-7.09 (1H, t) 3.69 (3H, s), 2.62 (3H, s), 2.55 (3H, s).

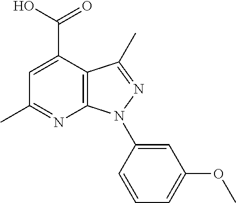

### IM54: 1-(3-Methoxy-phenyl)-3,6-dimethyl-1H-pyrazolo[3,4-b]pyridine-4-carboxylic acid

The crude compound was prepared from IM26 and subsequently washed with diethyl ether to give the title compound as a pale yellow powder (0.90 g, 62%). 1H NMR (DMSO-d6, 400 MHz) δ 13.92 (1H, br s), 7.89-7.84 (2H, m), 7.54 (1H, s), 7.47-7.43 (1H, m), 6.91-6.89 (1H, m), 3.84 (3H, s), 2.69 (3H, s), 2.66 (3H, s).

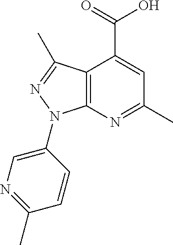 [SMILES: Cc1ccc(-n2nc(C)c3c(C(=O)O)cc(C)nc32)cn1]

### IM55: 3,6-Dimethyl-1-(6-methyl-pyridin-3-yl)-1H-pyrazolo[3,4-b]pyridine-4-carboxylic acid

The crude compound was prepared from IM27 and subsequently washed with water to give the title compound as a yellow solid (1.0 g, 74%). 1H NMR (DMSO-d6, 400 MHz) δ 13.96 (1H, br s), 9.27-9.28 (1H, d), 8.48-8.46 (1H, m), 7.57 (1H, s), 7.48-7.46 (1H, m), 2.72 (3H, s), 2.68 (3H, s), 2.56 (3H, s).

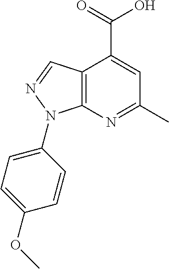 [SMILES: COc1ccc(-n2ncc3c(C(=O)O)cc(C)nc32)cc1]

### IM56: 1-(4-Methoxy-phenyl)-6-methyl-1H-pyrazolo[3,4-b]pyridine-4-carboxylic acid

The crude product was prepared from IM28 and subsequently washed with water followed by diethyl ether to give the title compound as a yellow solid (2.2 g, 71%). 1H NMR (DMSO-d6, 400 MHz) δ 13.95 (1H, broad), 8.51 (1H, s), 8.07-8.04 (2H, m), 7.68 (1H, s), 7.15-7.13 (2H, m), 3.83 (3H, s), 2.71 (3H, s).

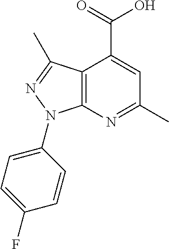 [SMILES: Cc1cc(C(=O)O)c2c(C)nn(-c3ccc(F)cc3)c2n1]

### IM57: 1-(4-Fluoro-phenyl)-3,6-dimethyl-1H-pyrazolo[3,4-b]pyridine-4-carboxylic acid

The crude product was prepared from IM29 and subsequently washed with water followed by diethyl ether to give the title compound as a yellow solid (3.0 g, 65%).

1H NMR (DMSO-d6, 300 MHz) δ 13.94 (1H, br s), 8.24-8.20 (2H, m), 7.54 (1H, s), 7.43-7.37 (2H, m), 2.68 (3H, s), 2.66 (3H, s).

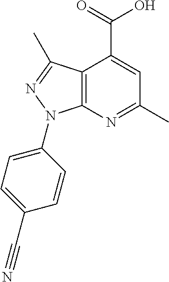 [SMILES: Cc1cc(C(=O)O)c2c(C)nn(-c3ccc(C#N)cc3)c2n1]

### IM58: 1-(4-Cyano-phenyl)-3,6-dimethyl-1H-pyrazolo[3,4-b]pyridine-4-carboxylic acid

The crude product was prepared from IM30 and subsequently washed with water followed by diethyl ether to give the title compound as a yellow solid (1.5 g, 55%).

1H NMR (DMSO-d6, 400 MHz) δ 8.59-8.56 (1H, m), 8.39-8.37 (1H, m), 8.05-7.98 (2H, m), 7.44-7.43 (1H, m), 2.68 (3H, s), 2.64 (3H, s).

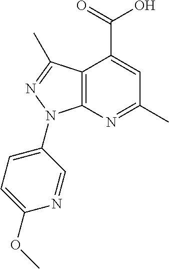 [SMILES: COc1ccc(-n2nc(C)c3c(C(=O)O)cc(C)nc32)cn1]

### IM59: 1-(6-Methoxy-pyridin-3-yl)-3,6-dimethyl-1H-pyrazolo[3,4-b]pyridine-4-carboxylic acid

The crude product was prepared from IM31 and subsequently washed with water followed by diethyl ether to give the title compound as a yellow solid (3.0 g, 83%). 1H NMR (DMSO-d6, 300 MHz) δ 12.0 (1H, br s), 8.96-8.95 (1H, d), 8.43-8.40 (1H, m), 7.21 (1H, s), 7.01-6.99 (1H, m), 3.91 (3H, s), 2.61 (3H, s), 2.60 (3H, s).

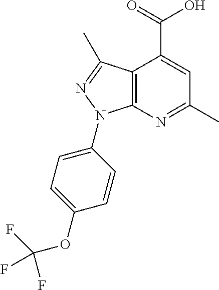 [SMILES: Cc1cc(C(=O)O)c2c(C)nn(-c3ccc(OC(F)(F)F)cc3)c2n1]

### IM60: 3,6-Dimethyl-1-(4-trifluoromethoxy-phenyl)-1H-pyrazolo[3,4-b]pyridine-4-carboxylic acid

The crude product was prepared from IM32 and subsequently washed with water followed by diethyl ether to give the title compound as a yellow solid (3.0 g, 65%). 1H NMR (DMSO-d6, 300 MHz) δ 13.97 (1H, br s), 8.39-8.35 (2H, m), 7.58-7.56 (3H, m), 2.70 (3H, s), 2.67 (3H, s).

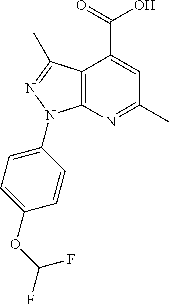 [SMILES: Cc1cc(C(=O)O)c2c(C)nn(-c3ccc(OC(F)F)cc3)c2n1]

### IM61: 1-(4-Difluoromethoxy-phenyl)-3,6-dimethyl-1H-pyrazolo[3,4-b]pyridine-4-carboxylic acid

The crude product was prepared from IM33 and subsequently washed with water followed by diethyl ether to give the title compound as a yellow solid (1.0 g, 72%). 1H NMR (DMSO-d6, 400 MHz) δ 13.09 (1H, br s), 8.26=8.24 (2H, m), 7.51 (1H, s), 7.46-7.09 (3H, m), 2.68 (3H, s), 2.65 (3H, s).

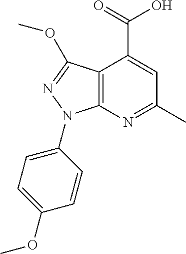

### IM62: 3-Methoxy-1-(4-methoxy-phenyl)-6-methyl-1H-pyrazolo[3,4-b]pyridine-4-carboxylic acid

Prepared from IM39 to give a solid (0.16 g, 89%) sufficiently pure for the next step. The hydrolysis was performed at room temperature.

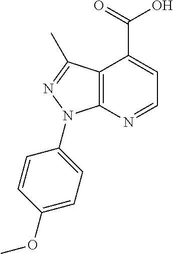 [SMILES: COc1ccc(-n2nc(C)c3c(C(=O)O)ccnc32)cc1]

### IM63: 1-(4-Methoxy-phenyl)-3-methyl-1H-pyrazolo[3,4-b]pyridine-4-carboxylic acid

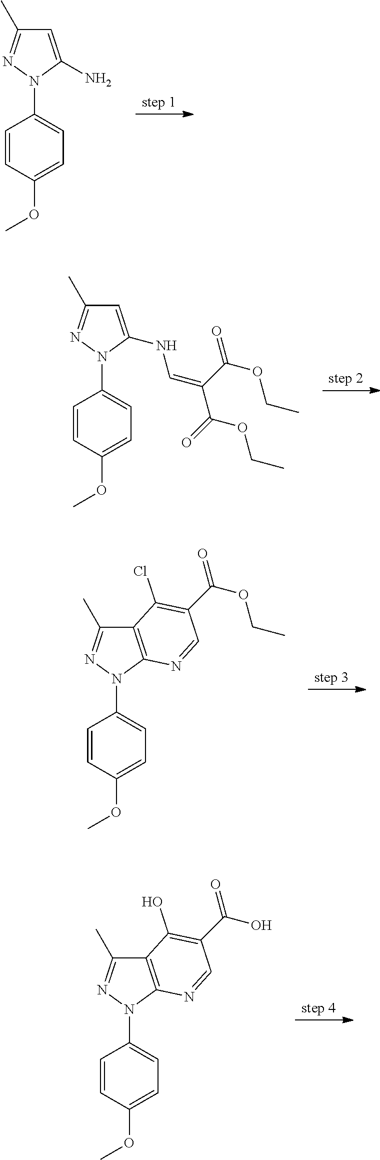

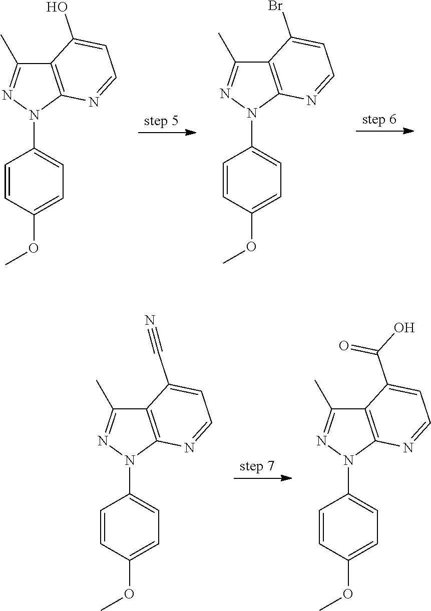 [SMILES: COc1ccc(-n2nc(C)c3c(O)ccnc32)cc1 ; COc1ccc(-n2nc(C)c3c(Br)ccnc32)cc1 ; COc1ccc(-n2nc(C)c3c(C#N)ccnc32)cc1 ; COc1ccc(-n2nc(C)c3c(C(=O)O)ccnc32)cc1]

Prepared analogously to derivatives described in Pharmadeutica Acta Helvetiae 71 (1996) 213-219 and references cited herein.

### Step 1:

To a solution of 2-(4-methoxy-phenyl)-5-methyl-2H-pyrazol-3-ylamine (1.0 g, 4.9 mmol) in EtOH (10 mL) was added diethyl 2-(ethoxymethylene)malonate (1.17 g, 5.4 mmol) at room temperature. The mixture was subsequently stirred at 80° C. for 12 h. The reaction mixture was cooled to room, temperature and the excess ethanol was removed under reduced pressure. The resulting mixture was poured into ice-cold 1N NaOH solution (20 mL). The mixture was extracted with EtOAc (2×20 mL). The combined organic layers were washed with water and evaporated to dryness. Flash chromatography (silica, 10% EtOAc in hexanes) gave 2-{[2-(4-methoxy-phenyl)-5-methyl-2H-pyrazol-3-ylamino]-methylene}-malonic acid diethyl ester as a solid (0.80 g, 43).

### Step 2:

A mixture of POCl₃ and 2-{[2-(4-methoxy-phenyl)-5-methyl-2H-pyrazol-3-ylamino]-methylene}-malonic acid diethyl ester (0.80 g, 2.13 mmol) was refluxed for 6 h. The reaction mixture was cooled to room temperature and then poured into ice-water. The mixture was extracted with methylene chloride (2×20 mL). The combined organic layers were washed with water and evaporated to dryness. The crude product was washed with diethyl ether to give crude 4-chloro-1-(4-methoxy-phenyl)-3-methyl-1H-pyrazolo[3,4-b]pyridine-5-carboxylic acid ethyl ester as a brown solid (0.40 g, 54%) sufficiently pure for the next step.

### Step 3:

To a solution of 4-chloro-1-(4-methoxy-phenyl)-3-methyl-1H-pyrazolo[3,4-b]pyridine-5-carboxylic acid ethyl ester (9.0 g, 26.0 mmol) in EtOH (100 mL) was added KOH (7.3 g, 130.0 mmol). The mixture was refluxed for 3 h. The reaction mixture was cooled to room temperature and then poured into ice-water. The mixture was acidified by addition of 10% HCl. The mixture was filtered and the remanens was washed with water, petroleum ether and then dried in vacuo to give crude 4-hydroxy-1-(4-methoxy-phenyl)-3-methyl-1H-pyrazolo[3,4-b]pyridine-5-carboxylic acid as a brown solid (4.2 g, 53%) sufficiently pure for the next step.

### Step 4:

A solution of 4-hydroxy-1-(4-methoxy-phenyl)-3-methyl-1H-pyrazolo[3,4-b]pyridine-5-carboxylic acid (4.2 g, 14.0 mmol) in diphenyl ether was heated to 180° C. for 3 h. The reaction mixture was cooled to room temperature, diluted with petroleum ether and then filtered. The remanens was washed with petroleum ether to give 1-(4-methoxy-phenyl)-3-methyl-1H-pyrazolo[3,4-b]pyridin-4-ol as a brown solid sufficiently pure for the next step (2.40 g, 61%). 1H NMR (DMSO-d6, 400 MHz) δ 11.57 (1H, s), 8.24-8.22 (1H, m), 8.10-8.08 (2H, m), 7.08-7.08 (2H, m), 6.59-6.58 (1H, m), 3.80 (3H, s), 2.61 (3H, s).

### Step 5:

To a solution 1-(4-methoxy-phenyl)-3-methyl-1H-pyrazolo[3,4-b]pyridin-4-ol (2.2 g, 8.6 mmol) in acetonitrile was added POBr₃ (4.90 g, 17.1 mmol) in small portions at room temperature. The reaction mixture was refluxed for 10 h. The reaction mixture was cooled to room temperature and then poured into ice-water. The mixture was extracted with EtOAc (2×100 mL). The combined organic layers were washed with water and evaporated to dryness to give crude 4-bromo-1-(4-methoxy-phenyl)-3-methyl-1H-pyrazolo[3,4-b]pyridine as a colorless solid (2.0 g, 74%) sufficiently pure for the next step. 1H NMR (DMSO-d6, 300 MHz) δ 8.42-8.40 (1H, m), 8.03-7.98 (2H, m), 7.59-7.57 (1H, m), 7.14-7.05 (2H, m), 3.82 (3H, s), 2.74 (3H, s).

### Step 6:

To solution of 4-bromo-1-(4-methoxy-phenyl)-3-methyl-1H-pyrazolo[3,4-b]pyridine (2.0 g, 6.3 mmol) in DMF was added CuCN (1.12 g, 12.6 mmol). The reaction mixture was stirred at 145° C. for 18 h. The reaction mixture was diluted with water and filtered through a plug of celite. The filtrate was extracted with methylene chloride (2×100 mL). The combined organic layers were washed with water and evaporated to dryness to give crude 1-(4-methoxy-phenyl)-3-methyl-1H-pyrazolo[3,4-b]pyridine-4-carbonitrile (1.5 g, 90%) sufficiently pure for the next step.

1H NMR (DMSO-d6, 400 MHz) δ: 8.83-8.82 (1H, m), 8.07-7.97 (2H, m), 7.85-7.84 (1H, m), 7.15-7.11 (2H, m), 3.82 (3H, s), 2.75 (3H, s).

### Step 7:

To solution of 1-(4-methoxy-phenyl)-3-methyl-1H-pyrazolo[3,4-b]pyridine-4-carbonitrile (1.2 g, 4.54 mmol) in EtOH (15 mL) was added NaOH (1.82 g, 45.5 mmol) and the reaction mixture was stirred for 24 h at 80° C. The reaction mixture was cooled to room temperature and then concentrated in vacuo. The mixture was diluted with water and acidified by addition of 10% HCl. The mixture was then extracted with methylene chloride (2×75 mL). The combined organic layers were washed with water and evaporated to dryness to give the title compound IM63 as an off-white solid (0.90 g, 70%). 1H NMR (DMSO-d6, 400 MHz) δ 13.95 (1H, s) 8.73-8.71 (1H, m), 8.04-8.01 (2H, m), 7.62-7.61 (1H, m), 7.13-7.10 (2H, m), 3.82 (3H, s), 2.69 (3H, s).

### Preparation of Examples

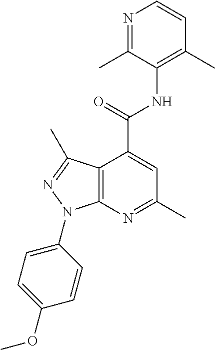

### 1: 1-(4-Methoxy-phenyl)-3,6-dimethyl-1H-pyrazolo[3,4-b]pyridine-4-carboxylic acid (2,4-dimethyl-pyridin-3-yl)-amide

A round bottomed flask was charged with 1-(4-methoxy-phenyl)-3,6-dimethyl-1H-pyrazolo[3,4-b]pyridine-4-carboxylic acid IM51 (800 mg, 2.69 mmol) in pyridine (10 mL). To this mixture was added 3-amino-2,4-dimethylpyridine (427 mg, 3.50 mmol) and POCl₃ (618 mg, 4.04 mmol) and the resulting mixture was slowly heated to 100° C. and stirred at this temperature for 2 h. The reaction mixture was cooled to room temperature and then poured into ice-cold water (30 mL). The mixture was extracted with EtOAc (2×40 mL). The combined organic layers were washed with brine, dried over anhydrous Na₂SO₄ and evaporated to dryness. Flash chromatography (silica, 80% EtOAc in hexanes) gave the title compound as a solid (260 mg, 24%). mp=224-227° C. 1H NMR (400 MHz, DMSO-d6) δ 10.40 (1H, s), 8.30-8.29 (1H, d), 8.07-8.04 (m, 2H), 7.48 (1H, br), 7.23-7.22 (1H, d), 7.14-7.12 (m, 2H), 3.83 (3H, s), 2.73 (3H, s), 2.56 (3H, s), 2.50 (3H, s), 2.32 (3H, s).

LC-MS (m/z) 402.2 (MH⁺); t_{R}=0.51.

The following compounds were prepared analogously:

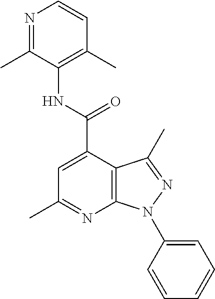 [SMILES: Cc1cc(C(=O)Nc2c(C)ccnc2C)c2c(C)nn(-c3ccccc3)c2n1]

### 2: 3,6-Dimethyl-1-phenyl-1h-pyrazolo[3,4-b]pyridine-4-carboxylic acid (2,4-dimethyl-pyridin-3-yl)-amide

Using 3,6-dimethyl-1-phenyl-1h-pyrazolo[3,4-b]pyridine-4-carboxylic acid IM43 (1.09 g, 4.08 mmol), 3-amino-2,4-dimethylpyridine (0.498 g, 4.08 mmol), pyridine (20 mL) and phosphoryl chloride (0.688 g, 4.48 mmol). Flash chromatography (silica, EtOAc/heptane 1:2) gave the title compound as a solid (0.35 g, 23%).

LC-MS (m/z) 372.2 (MH⁺); t_{R}=0.54.

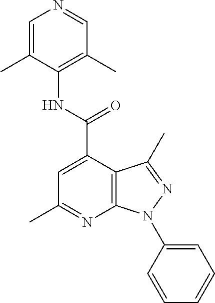 [SMILES: Cc1cc(C(=O)Nc2c(C)cncc2C)c2c(C)nn(-c3ccccc3)c2n1]

### 3: 3,6-Dimethyl-1-phenyl-1H-pyrazolo[3,4-b]pyridine-4-carboxylic acid (3,5-dimethyl-pyridin-4-yl)-amide

Using 3,6-dimethyl-1-phenyl-1h-pyrazolo[3,4-b]pyridine-4-carboxylic acid IM43 (1.75 g, 6.55 mmol), 3,5-dimethylpyridin-4-amine (0.800 g, 6.55 mmol), pyridine (30 mL) and phosphoryl chloride (0.671 mL, 7.20 mmol). Flash chromatography (silica, EtOAc/heptane 1:1) gave the title compound as a solid (0.24 g, 10%).

LC-MS (m/z) 372.2 (MN*); t_{R}=0.56.

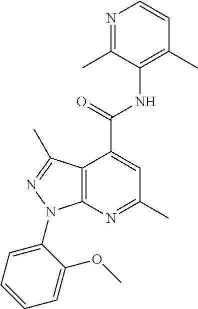 [SMILES: COc1ccccc1-n1nc(C)c2c(C(=O)Nc3c(C)ccnc3C)cc(C)nc21]

### 4: 1-(2-Methoxy-phenyl)-3,6-dimethyl-1H-pyrazolo[3,4-b]pyridine-4-carboxylic acid (2,4-dimethyl-pyridin-3-A-amide

Using 1-(2-Methoxy-phenyl)-3,6-dimethyl-1H-pyrazolo[3,4-b]pyridine-4-carboxylic acid IM53 (0.90 g, 3.03 mmol), 2,4-Dimethyl-pyridin-3-ylamine (0.37 g, 3.03 mmol), pyridine (15 mL) and phosphoryl chloride (0.55 g, 4.57 mmol). Flash chromatography (silica, EtOAc/heptane 4:1) gave the title compound as a solid (0.48 g, 39%).

LC-MS (m/z) 402.2 (MH⁺); t_{R}=0.43.

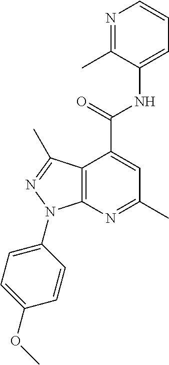 [SMILES: COc1ccc(-n2nc(C)c3c(C(=O)Nc4cccnc4C)cc(C)nc32)cc1]

### 5: 1-(4-Methoxy-phenyl)-3,6-dimethyl-1H-pyrazolo[3,4-b]pyridine-4-carboxylic acid (2-methyl-pyridin-3-yl)-amide

Using 1-(4-methoxy-phenyl)-3,6-dimethyl-1H-pyrazolo[3,4-b]pyridine-4-carboxylic acid IM51 (0.50 g, 1.68 mmol), 2-Methyl-pyridin-3-ylamine (0.181 g, 1.68 mmol), pyridine (8) and phosphoryl chloride (0.387 g, 2.46 mmol). Flash chromatography (silica, EtOAc/heptane 4:1) gave the title compound as a solid (0.20 g, 30%).

LC-MS (m/z) 388.2 (WO; t_{R}=0.51.

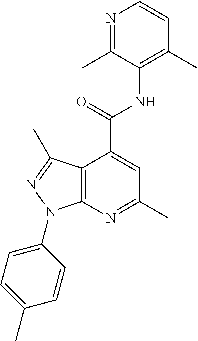 [SMILES: Cc1ccc(-n2nc(C)c3c(C(=O)Nc4c(C)ccnc4C)cc(C)nc32)cc1]

### 6: 3,6-Dimethyl-1-p-tolyl-1H-pyrazolo[3,4-b]pyridine-4-carboxylic acid (2,4-dimethyl-pyridin-3-yl)-amide

Using 3,6-Dimethyl-1-p-tolyl-1H-pyrazolo[3,4-b]pyridine-4-carboxylic acid IM52 (1.00 g, 3.55 mmol), 2,4-Dimethyl-pyridin-3-ylamine (0.43 g, 3.52 mmol), pyridine (15 mL) and phosphoryl chloride (0.60 g, 3.95 mmol). Washing the crude product with diethyl ether gave the title compound as a solid (0.52 g, 37%).

LC-MS (m/z) 386.2 (MH⁺); t_{R}=0.58.

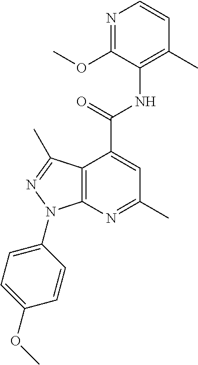 [SMILES: COc1ccc(-n2nc(C)c3c(C(=O)Nc4c(C)ccnc4OC)cc(C)nc32)cc1]

### 7: 1-(4-Methoxy-phenyl)-3,6-dimethyl-1H-pyrazolo[3,4-b]pyridine-4-carboxylic acid (2-methoxy-4-methyl-pyridin-3-yl)-amide

Using 1-4-methoxy-phenyl)-3,6-dimethyl-1H-pyrazolo[3,4-b]pyridine-4-carboxylic acid IM51 (0.50 g, 1.68 mmol), 2-Methoxy-4-methyl-pyridin-3-ylamine IM01 (0.240 g, 1.74 mmol), pyridine (8) and phosphoryl chloride (0.387 g, 2.52 mmol). Flash chromatography (silica, EtOAc/heptane 4:1) gave the title compound as a solid (0.13 g, 19%).

LC-MS (m/z) 418.2 (MH⁺); t_{R}=0.73.

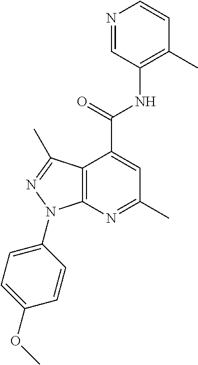 [SMILES: COc1ccc(-n2nc(C)c3c(C(=O)Nc4cnccc4C)cc(C)nc32)cc1]

### 8: 1-(4-Methoxy-phenyl)-3,6-dimethyl-1H-pyrazolo[3,4-b]pyridine-4-carboxylic acid (4-methyl-pyridin-3-yl)-amide

Using 1-(4-methoxy-phenyl)-3,6-dimethyl-1H-pyrazolo[3,4-b]pyridine-4-carboxylic acid IM51 (0.50 g, 1.68 mmol), 4-Methyl-pyridin-3-ylamine (0.181 g, 1.68 mmol), pyridine (8) and phosphoryl chloride (0.387 g, 2.46 mmol). Flash chromatography (silica, EtOAc/heptane 4:1) gave the title compound as a solid (0.20 g, 30%).

LC-MS (m/z) 388.2 (MH⁺); t_{R}=0.52.

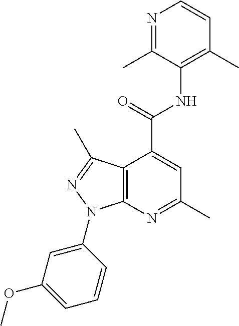

### 9: 1-(3-Methoxy-phenyl)-3,6-dimethyl-1H-pyrazolo[3,4-b]pyridine-4-carboxylic acid (2,4-dimethyl-pyridin-3-yl)-amide

Using 1-(3-Methoxy-phenyl)-3,6-dimethyl-1H-pyrazolo[3,4-b]pyridine-4-carboxylic acid IM54, 2,4-Dimethyl-pyridin-3-ylamine (0.37 g, 3.03 mmol), pyridine (15 mL) and phosphoryl chloride (0.55 g, 3.63 mmol). Flash chromatography (silica, EtOAc/heptane 4:1) gave the title compound as a solid (0.697 g, 58%).

LC-MS (m/z) 402.2 (MH⁺); t_{R}=0.54.

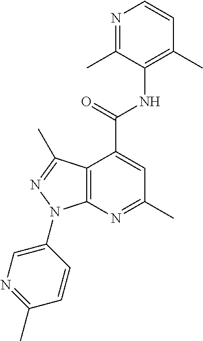 [SMILES: Cc1ccc(-n2nc(C)c3c(C(=O)Nc4c(C)ccnc4C)cc(C)nc32)cn1]

### 10: 3,6-Dimethyl-1-(6-methyl-pyridin-3-yl)-1H-pyrazolo[3,4-b]pyridine-4-carboxylic acid (2,4-dimethyl-pyridin-3-yl)-amide

Using 3,6-Dimethyl-1-(6-methyl-pyridin-3-yl)-1H-pyrazolo[3,4-b]pyridine-4-carboxylic acid IM55 (1.00 g, 3.54 mmol), 2,4-Dimethyl-pyridin-3-ylamine (0.43 g, 3.54 mmol), pyridine (15 mL) and phosphoryl chloride (0.39 mL, 4.25 mmol). Flash chromatography (silica, EtOAc/heptane 4:1) gave the title compound as a colorless solid (0.400 g, 29%).

LC-MS (m/z) 387.2 (MH⁺); t_{R}=0.32.

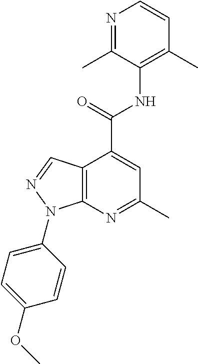 [SMILES: COc1ccc(-n2ncc3c(C(=O)Nc4c(C)ccnc4C)cc(C)nc32)cc1]

### 11: 1-(4-Methoxy-phenyl)-6-methyl-1H-pyrazolo[3,4-b]pyridine-4-carboxylic acid (2,4-dimethyl-pyridin-3-yl)-amide

Prepared using IM56 and 3-amino-2,4-dimethylpyridine.

LC-MS (m/z) 388.2 (MH⁺); t_{R}=0.51.

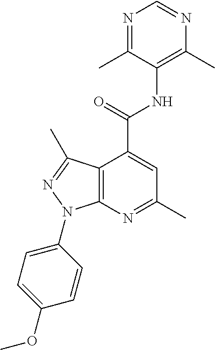

### 12: 1-(4-Methoxy-phenyl)-3,6-dimethyl-1H-pyrazolo[3,4-b]pyridine-4-carboxylic acid (4,6-dimethyl-pyrimidin-5-yl)-amide

Prepared using IM56 and IM06.

LC-MS (m/z) 403.2 (MH⁺); t_{R}=0.62.

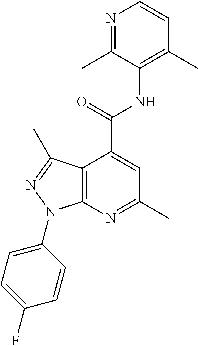 [SMILES: Cc1cc(C(=O)Nc2c(C)ccnc2C)c2c(C)nn(-c3ccc(F)cc3)c2n1]

### 13: 1-(4-Fluoro-phenyl)-3,6-dimethyl-1H-pyrazolo[3,4-b]pyridine-4-carboxylic acid (2,4-dimethyl-pyridin-3-yl)-amide

Prepared using IM57 and 3-amino-2,4-dimethylpyridine.

LC-MS (m/z) 390.2 (MH⁺); t_{R}=0.56.

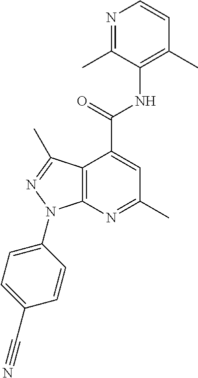 [SMILES: Cc1cc(C(=O)Nc2c(C)ccnc2C)c2c(C)nn(-c3ccc(C#N)cc3)c2n1]

### 14: 1-(4-Cyano-phenyl)-3,6-dimethyl-1H-pyrazolo[3,4-b]pyridine-4-carboxylic acid (2,4-dimethyl-pyridin-3-yl)-amide

Prepared using IM58 and 3-amino-2,4-dimethylpyridine.

LC-MS (m/z) 397.2 (MH⁺); t_{R}=0.52.

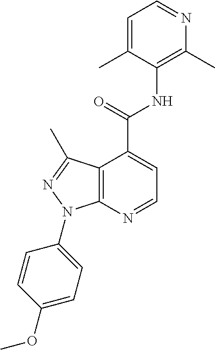

### 15: 1-(4-Methoxy-phenyl)-3-methyl-1H-pyrazolo[3,4-b]pyridine-4-carboxylic acid (2,4-dimethyl-pyridin-3-yl)-amide

Prepared using IM63 and 3-amino-2,4-dimethylpyridine.

LC-MS (m/z) 388.2 (MH⁺); t_{R}=0.48.

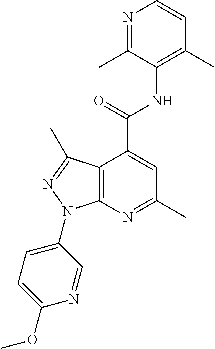

### 16: 1-(6-Methoxy-pyridin-3-yl)-3,6-dimethyl-1H-pyrazolo[3,4-b]pyridine-4-carboxylic acid (2,4-dimethyl-pyridin-3-yl)-amide

Prepared using IM59 and 3-amino-2,4-dimethylpyridine.

LC-MS (m/z) 403.2 (MH⁺); t_{R}=0.48.

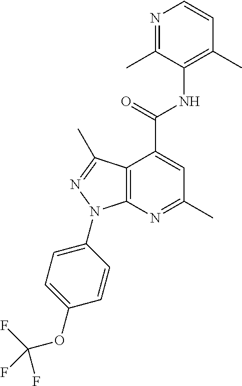 [SMILES: Cc1cc(C(=O)Nc2c(C)ccnc2C)c2c(C)nn(-c3ccc(OC(F)(F)F)cc3)c2n1]

### 17: 3,6-Dimethyl-1-(4-trifluoromethoxy-phenyl)-1H-pyrazolo[3,4-b]pyridine-4-carboxylic acid (2,4-dimethyl-pyridin-3-yl)-amide

Prepared using IM60 and 3-amino-2,4-dimethylpyridine.

LC-MS (m/z) 456.2 (MH⁺); t_{R}=0.68.

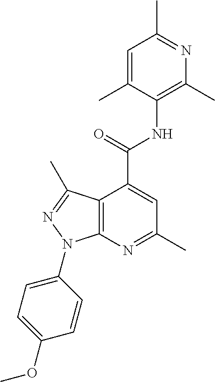

### 18: 1-(4-Methoxy-phenyl)-3,6-dimethyl-1H-pyrazolo[3,4-b]pyridine-4-carboxylic acid (2,4,6-trimethyl-pyridin-3-yl)-amide

Prepared using IM51 and 2,4,6-trimethyl-pyridin-3-ylamine.

LC-MS (m/z) 416.2 (MH⁺); t_{R}=0.52.

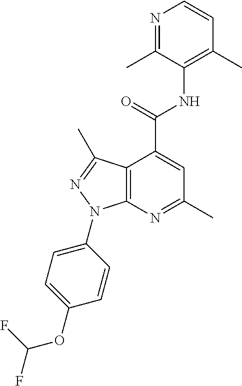 [SMILES: Cc1cc(C(=O)Nc2c(C)ccnc2C)c2c(C)nn(-c3ccc(OC(F)F)cc3)c2n1]

### 19: 1-(4-Difluoromethoxy-phenyl)-3,6-dimethyl-1H-pyrazolo[3,4-b]pyridine-4-carboxylic acid (2,4-dimethyl-pyridin-3-O-amide

Prepared using IM61 and 2,6-dimethyl-pyridin-3-ylamine.

LC-MS (m/z) 438.2 (MH⁺); t_{R}=0.58.

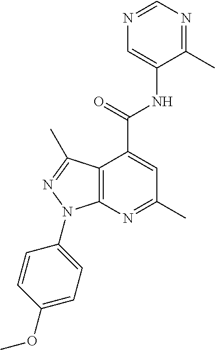

### 20: 1-(4-Methoxy-phenyl)-3,6-dimethyl-1H-pyrazolo[3,4-b]pyridine-4-carboxylic acid (4-methyl-pyrimidin-5-A-amide

Prepared using IM51 and 4-methyl-pyrimidin-5-ylamine.

LC-MS (m/z) 389.2 (MH⁺); t_{R}=0.62.

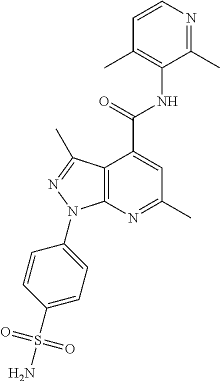 [SMILES: Cc1cc(C(=O)Nc2c(C)ccnc2C)c2c(C)nn(-c3ccc(S(N)(=O)=O)cc3)c2n1]

### 21: 3,6-Dimethyl-1-(4-sulfamoyl-phenyl)-1H-pyrazolo[3,4-b]pyridine-4-carboxylic acid (2,4-dimethyl-pyridin-3-yl)-amide

Prepared using IM46 and 3-amino-2,4-dimethylpyridine.

LC-MS (m/z) 451.2 (MH⁺); t_{R}=0.37.

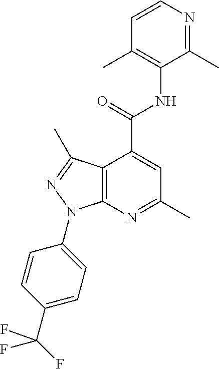 [SMILES: Cc1cc(C(=O)Nc2c(C)ccnc2C)c2c(C)nn(-c3ccc(C(F)(F)F)cc3)c2n1]

### 22: 3,6-Dimethyl-1-(4-trifluoromethyl-phenyl)-1H-pyrazolo[3,4-b]pyridine-4-carboxylic acid (2,4-dimethyl-pyridin-3-yl)-amide

Prepared using IM47 and 3-amino-2,4-dimethylpyridine.

LC-MS (m/z) 440.2 (MH⁺); t_{R}=0.67.

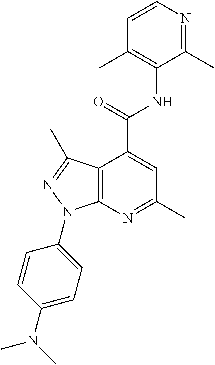

### 23: 1-(4-Dimethylamino-phenyl)-3,6-dimethyl-1H-pyrazolo[3,4-b]pyridine-4-carboxylic acid (2,4-dimethyl-pyridin-3-yl)-amide

Prepared using IM48 and 3-amino-2,4-dimethylpyridine, a catalytic amount of DMAP was included in the reaction mixture.

LC-MS (m/z) 415.2 (MH⁺); t_{R}=0.37.

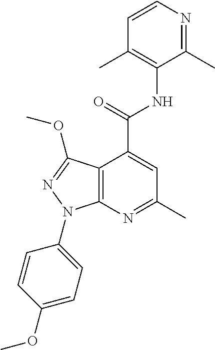

### 24: 3-Methoxy-1-(4-methoxy-phenyl)-6-methyl-1H-pyrazolo[3,4-b]pyridine-4-carboxylic acid (2,4-dimethyl-pyridin-3-yl)-amide

Prepared using IM62 and 3-amino-2,4-dimethylpyridine.

LC-MS (m/z) 418.2 (MH⁺); t_{R}=0.56.

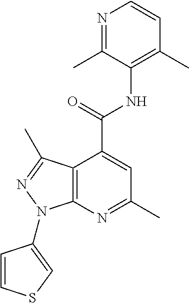 [SMILES: Cc1cc(C(=O)Nc2c(C)ccnc2C)c2c(C)nn(-c3ccsc3)c2n1]

### 25: 3,6-Dimethyl-1-thiophen-3-yl-1H-pyrazolo[3,4-b]pyridine-4-carboxylic acid (2,4-dimethyl-pyridin-3-yl)-amide

Prepared using IM49 and 3-amino-2,4-dimethylpyridine.

LC-MS (m/z) 378.1 (MH⁺); t_{R}=0.53.

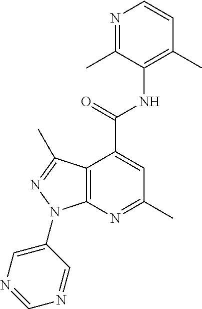 [SMILES: Cc1cc(C(=O)Nc2c(C)ccnc2C)c2c(C)nn(-c3cncnc3)c2n1]

### 26: 3,6-Dimethyl-1-pyrimidin-5-yl-1H-pyrazolo[3,4-b]pyridine-4-carboxylic acid (2,4-dimethyl-pyridin-3-yl)-amide

Prepared using IM50 and 3-amino-2,4-dimethylpyridine.

LC-MS (m/z) 374.4 (MK); t_{R}=0.38.

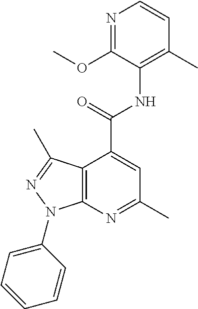 [SMILES: COc1nccc(C)c1NC(=O)c1cc(C)nc2c1c(C)nn2-c1ccccc1]

### 27: 3,6-Dimethyl-1-phenyl-1H-pyrazolo[3,4-b]pyridine-4-carboxylic acid (2-methoxy-4-methyl-pyridin-3-yl)-amide

A round bottomed flask was charged with 3,6-dimethyl-1-phenyl-1h-pyrazolo[3,4-b]pyridine-4-carboxylic acid IM43 (1.2 g, 4.49 mmol), 3-amino-4-methyl-2-methoxy pyridine (620 mg, 4.49 mmol) and DIPEA (2.94 mL, 8.99 mmol) in DMF (50 mL) at room temperature. To this mixture was added HATU (2.04 g, 5.39 mmol) and the resulting mixture was stirred for 16 h. The mixture was then poured into water (100 mL). The mixture was extracted with ethyl acetate (2×200 mL). The combined organic layers were washed with brine, dried over anhydrous Na₂SO₄ and evaporated to dryness. Flash chromatography (silica, 60% EtOAc in petroleum-ether) gave the title compound as a solid (600 mg, 34%). mp=172-174° C. 1H NMR (400 MHz, DMSO-d6) δ 10.20 (1H, s), 8.27-8.25 (2H, d), 8.03-8.02 (1H, d), 7.58-7.54 (2H, m), 7.41 (1H, s), 7.35-7.31 (1H, m), 6.99-6.98 (1H, m), 3.90 (3H, s), 2.73 (3H, s), 2.60 (3H, s), 2.31 (3H, s).

LC-MS (m/z) 388.2 (MH⁺); t_{R}=0.77.

The following compounds were prepared analogously:

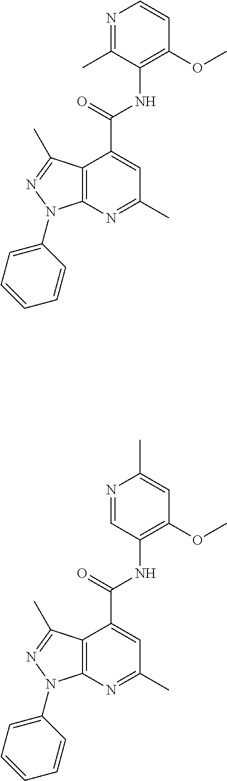 [SMILES: COc1ccnc(C)c1NC(=O)c1cc(C)nc2c1c(C)nn2-c1ccccc1 ; COc1cc(C)ncc1NC(=O)c1cc(C)nc2c1c(C)nn2-c1ccccc1]

### 28: 3,6-Dimethyl-1-phenyl-1H-pyrazolo[3,4-b]pyridine-4-carboxylic acid (4-methoxy-2-methyl-pyridin-3-yl)-amide and

### 29: 3,6-Dimethyl-1-phenyl-1H-pyrazolo[3,4-b]pyridine-4-carboxylic acid (4-methoxy-6-methyl-pyridin-3-yl)-amide

Using 3,6-dimethyl-1-phenyl-1 h-pyrazolo[3,4-b]pyridine-4-carboxylic acid IM43 (1.2 g, 4.49 mmol), a mixture of regioisomers 4-methoxy-2-methyl-pyridin-3-ylamine and 4-methoxy-6-methyl-pyridin-3-ylamine, IM04+IM05, (620 mg as a mixture, 4.49 mmol), DIPEA (2.94 mL, 8.99 mmol) and HATU (2.05 g, 5.39 mmol) in DMF (50 mL). Purification by preparative HPLC gave the two title compounds as solids.

28: (237 mg, 14%), mp=206-207° C. 1H NMR (400 MHz, DMSO-d6) δ 10.18 (1H, s), 8.33-8.32 (1H, d), 8.27-8.25 (2H, d), 7.58-7.54 (2H, m), 7.40 (1H, s), 7.35-7.31 (1H, m), 7.07-7.05 (1H, d), 3.89 (3H, s), 2.73 (3H, s), 2.60 (3H, s), 2.45 (3H, s).

LC-MS (m/z) 388.2 (MH⁺); t_{R}=0.52.

29: (87 mg, 5%), mp=230-232° C. 1H NMR (400 MHz, DMSO-d6) δ 10.19 (1H, s), 8.57 (1H, s), 827-8.25 (2H, d), 7.58-7.50 (2H, m), 7.38-7.30 (m, 2H), 7.07 (1H, s), 3.00 (3H, s), 2.71 (3H, s), 2.56 (3H, s), 2.47 (3H, s).

LC-MS (m/z) 388.2 (MH⁺); t_{R}=0.54.

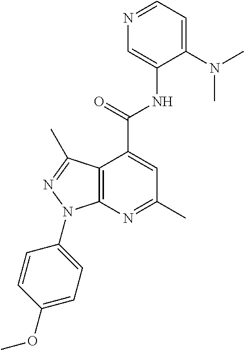

30: 1-(4-Methoxy-phenyl)-3,6-dimethyl-1H-pyrazolo[3,4-b]pyridine-4-carboxylic acid (4-dimethylamino-pyridin-3-yl)-amide

Prepared using IM51 and N⁴,N⁴-dimethylpyridine-3,4-diamine

LC-MS (m/z) 417.2 (MH⁺); t_{R}=0.50.

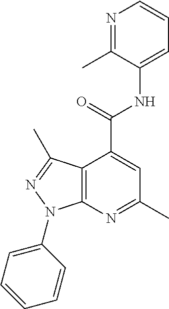 [SMILES: Cc1cc(C(=O)Nc2cccnc2C)c2c(C)nn(-c3ccccc3)c2n1]

31: 3,6-Dimethyl-1-phenyl-1H-pyrazolo[3,4-b]pyridine-4-carboxylic acid (2-methyl-pyridin-3-yl)-amide

A round bottomed flask was charged with 3,6-dimethyl-1-phenyl-1h-pyrazolo[3,4-b]pyridine-4-carboxylic acid IM43 (1.2 g, 4.49 mmol), 3-amino-2-methylpyridine (485 mg, 4.49 mmol) and N-methyl morpholine (907 mg, 8.99 mmol) in DMF (50 mL) at room temperature. To this mixture was added EDC.HCl (947 mg, 4.94 mmol) and HOBt (606 mg, 4.49 mmol). The mixture stirred at for 16 h. The reaction mixture was then poured into water (100 mL). The resulting mixture was extracted with ethyl acetate (2×200 mL). The combined organic layers were washed with brine, dried over anhydrous Na₂SO₄ and evaporated to dryness. Flash chromatography (silica, 70% EtOAc in petroleum-ether) gave the title compound as, a solid (277 mg, 17%). mp=208-209° C. 1H NMR (400 MHz, DMSO-d6) δ 10.48 (1H, s), 8.39-8.38 (1H, m), 8.27-8.25 (2H, d), 7.93-7.91 (1H, m), 7.59-7.55 (2H, m), 7.49 (1H, s), 7.35-7.32 (2H, m), 2.74 (3H, s), 2.58 (3H, s), 2.52 (3H, s).

LC-MS (m/z) 358.2 (MH⁺); t_{R}=0.53.

The following compounds were prepared analogously:

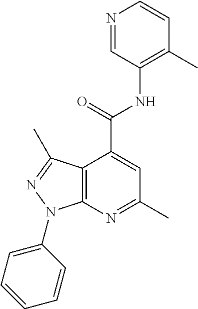 [SMILES: Cc1cc(C(=O)Nc2cnccc2C)c2c(C)nn(-c3ccccc3)c2n1]

### 32: 3,6-Dimethyl-1-phenyl-1H-pyrazolo[3,4-b]pyridine-4-carboxylic acid (4-methyl-pyridin-3-yl)-amide

Using 3,6-dimethyl-1-phenyl-1h-pyrazolo[3,4-b]pyridine-4-carboxylic acid IM43 (500 mg, 1.87 mmol), 3-amino-4-methylpyridine (202 mg, 1.87 mmol), N-methyl morpholine (378 mg, 3.74 mmol), EDC.HCl (394 mg, 2.06 mmol) and HOBt (252 mg, 1.87 mmol) in DMF (20 mL). Flash chromatography (silica, 80% EtOAc in petroleum-ether) gave the title compound as a solid (180 mg, 27%). mp=227-228° C. 1H NMR (400 MHz, DMSO-d6) δ 10.52 (1H, s), 8.64 (1H, s), 8.37-8.36 (1H, d), 8.27-8.25 (2H, m), 7.59-7.55 (2H, m), 7.51 (1H, s), 7.38-7.32 (2H, m), 2.74 (3H, s), 2.59 (3H, s), 2.34 (3H, s).

LC-MS (m/z) 358.0 (MH⁺).

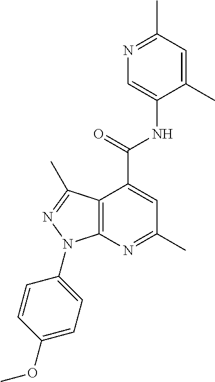

### 33: 1-(4-Methoxy-phenyl)-3,6-dimethyl-1H-pyrazolo[3,4-b]pyridine-4-carboxylic acid (4,6-dimethyl-pyridin-3-yl)-amide

To a suspension of 1-(4-methoxy-phenyl)-3,6-dimethyl-1H-pyrazolo[3,4-b]pyridine-4-carboxylic acid IM51 (1.60 g, 5.38 mmol), 5-amino-2,4-dimethylpyridine (786 mg, 6.43 mmol) and triethylamine (3.00 mL, 21.5 mmol) in THF (20 mL) was added a 50% (w/w) solution of 1-propanephosphonic acid cyclic anhydride in THF (6.84 g, 10.7 mmol) drop wise over 10 min. The mixture was stirred at room temperature Overnight. The mixture was poured into water (20 mL). The THF was removed in vacuo. Aqueous NH₃ solution was added to adjust pH to approximately 10 EtOH (25 mL) was added and the mixture was briefly heated to reflux and then left at room temperature overnight. The mixture was then cooled on an ice-water bath for 45 min and then filtered. The remanens was washed with water and dried in vacuo to give the title compound (1.41 g, 67%). 1H NMR (600 MHz, DMSO) δ 10.43 (s, 1H), 8.47 (s, 1H), 8.09-8.04 (m, 2H), 7.46 (s, 1H), 7.23 (s, 1H), 7.16-7.11 (m, 2H), 3.83 (s, 3H), 2.71 (s, 3H), 2.57 (s, 3H), 2.46 (s, 3H), 2.29 (s, 3H).

LC-MS (m/z) 402.2 (MH⁺); t_{R}=0.52.

The following compounds were prepared analogously:

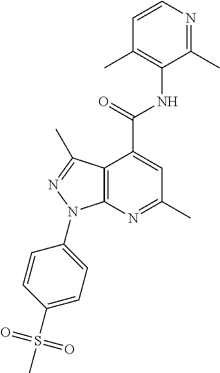 [SMILES: Cc1cc(C(=O)Nc2c(C)ccnc2C)c2c(C)nn(-c3ccc(S(C)(=O)=O)cc3)c2n1]

### 34: 1-(4-Methanesulfonyl-phenyl)-3,6-dimethyl-1H-pyrazolo[3,4-b]pyridine-4-carboxylic acid (2,4-dimethyl-pyridin-3-A-amide

Prepared using IM45 and 2,6-dimethyl-pyridin-3-ylamine.

LC-MS (m/z) 450.2 (MH⁺); t_{R}=0.43.

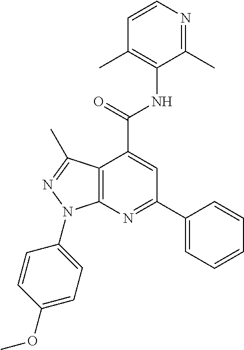 [SMILES: COc1ccc(-n2nc(C)c3c(C(=O)Nc4c(C)ccnc4C)cc(-c4ccccc4)nc32)cc1]

### 35: 1-(4-Methoxy-phenyl)-3-methyl-6-phenyl-1H-pyrazolo[3,4-b]pyridine-4-carboxylic acid (2,4-dimethyl-pyridin-3-yl)-amide

To a solution of IM44 (200 mg, 0.56 mmol) in 1 mL of DMF was added SOCl₂ (5 ml). The resulting mixture was heated at 100° C. for 1 h. The mixture was then evaporated to dryness to give the crude acid chloride (0.23 g).

The a separate solution of 3-amino-2,4-dimethylpyridine (68 mg, 0.56 mmol) in THF (5 mL) was added NaH (23 mg, 0.58 mmol) in small portions. The mixture was stirred at 25° C. for 30 min. The crude acid-chloride (0.23 g) was added to this solution. The resulting mixture was kept at 25° C. for 2 h. The mixture was poured into ice water and extracted with EtOAt (100 mL). The combined organic layers were dried over anhydrous Na₂SO₄ and evaporated to dryness. Flash chromatography (silica, petroleum ether:EtOAc=3:1) to give the title compound as a solid (172 mg, 67%). 1H NMR (CDCl3 400 MHz) δ 8.39 (m, 1H), 8.13-8.18 (m, 4 H), 7.82 (s, 1H)_{;} 7.51-7.56 (m 4H), 7.14 (d, 1H), 7.06-7.08 (m, 2H), 3.88 (s, 3H), 2.70 (s, 3 H), 2.64 (s, 3H), 2.43 (s, 3H).

LC-MS (m/z) 464.2 (MH⁺); t_{R}=0.66.

The following compounds were prepared analogously:

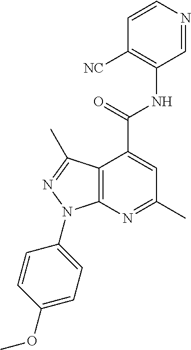

### 36: 1-(4-Methoxy-phenyl)-3,6-dimethyl-1H-pyrazolo[3,4-]pyridine-4-carboxylic acid (4-cyano-pyridin-3-yl)-amide

Prepared using IM51 and 3-Amino-isonicotinonitrile. LC-MS (m/z) 399.2 (MH⁺); t_{R}=0.68.

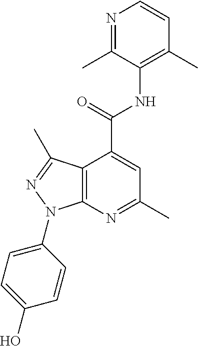 [SMILES: Cc1cc(C(=O)Nc2c(C)ccnc2C)c2c(C)nn(-c3ccc(O)cc3)c2n1]

### 37: 1-(4-Hydroxy-phenyl)-3,6-dimethyl-1H-pyrazolo[3,4-b]pyridine-4-carboxylic acid (2,4-dimethyl-pyridin-3-yl)-amide

To a solution of 1 (6.0 g, 15.0 mmol) in methylene chloride (70 mL) was added BBr₃ (18 mL, 18.0 mmol) at −78° C. The resulting mixture was slowly heated to room temperature and kept at this temperature for 18 h. The reaction mixture was quenched with MeOH (15 mL) and diluted with water. The mixture was basified by the addition of Na₂CO₃ then filtered. The remanens was washed with water and dried to give the title compound as a pale yellow solid (4.5 g, 78%). 1H NMR (DMSO-d6, 400 MHz, TMS) δ 10.39 (1H, s), 9.62 (1H, s). 8.30-8.28 (1H, m), 7.89-7.87 (2H, m), 7.46 (1H, s), 7.23-7.22 (1H, m), 6.94-6.92 (2H, m), 2.71 (3H, s), 2.55 (3H, s), 2.50 (3H, s), 2.32 (3H, s).

LC-MS (m/z) 388.2 (MH⁺); t_{R}=0.37.

### In Vitro Assays

The nicotinic acetylcholine receptor α7 is a calcium-permeable ion channel, whose activity can be measured by over expression in mammalian cells or oocytes. These two individual assays are described in Examples 2 and 3, respectively.

### Example 2

### α7 NNR Flux Assay

The nicotinic acetylcholine receptor α7 is a calcium-permeable ion channel, whose activity can be measured by over expression in mammalian cells or oocytes. In this version of the assay, the human α7 receptor is stably expressed in the rat GH4C1 cell line. The assay was used to identify positive allosteric modulators (PAMs) of the α7 receptor. Activation of the channel was measured by loading cells with the calcium-sensitive fluorescent dye. Calcium-4 (Assay kit from Molecular Devices), and then measuring real-time changes, in fluorescence upon treatment with test compounds.

The cell line ChanClone GH4C1-nAChRalpha7 from Genionics was seeded from frozen stock in 384-well plates in culture media 2-3 days before experiment to form an approximately 80% confluent layer on the day of experiment.

### Cell Plating and Dye Loading.

The cell culture were split into “22.5 cm×22.5 cm”-plates with approximately 100×10³ cells/cm². After four days incubation in a humidified incubator at 37° C. and 5% CO₂, it had grown to an 80-90% confluent layer, and the cells were harvested.

### Culture Media:

500 mL DMEM/F12 (Gibco 31331)

50 mL FBS (Gibco 10091-155, lot 453269FD)

5 mL Sodium Pyruvate (Gibco 11360)

5 mL Pen/Strep (Gibco 15140)

0.1 mg/mL G-418 (Gibco 11811-064)

Two or three days before the experiment the cells were seeded in 384 well plates from Greiner bio-one (781946, CELLCOAT, Poly-D-Lysine, black, μClear).

The media was poured off and the plate washed with PBS and left to drain. 5 mL Trypsin was added, cells were washed and incubated (at room temperature) for about 10 seconds. Trypsin was poured of quickly and the cells were incubated for 2 minutes at 37° C. (if the cells were not already detached). Cells were resuspended in 10 mL culture media and transferred to 50 mL tubes.

The cell suspension was counted (NucleoCounter, total cell count) from the first plates to estimate the total cell number of the whole batch.

The cells were seeded in 384 well plates with 30 μL/well (30000 cells/well) while stirring the cell suspension or otherwise preventing the cells from precipitating.

The plates were incubated at room temperature for 30-45 minutes.

The plates were placed in incubator for two days (37° C. and 5% CO₂).

### Loading the Cells

The loading buffer was 5% v/v Calcium-4 Kit and 2.5 mM Probenecid in assay buffer.

190 mL assay buffer

10 mL Kit-solution

2 mL 250 mM Probenecid

This volume was enough for 3×8 cell plates.

Culture media was removed from the cell plates and 20 μL loading buffer was added in each well. The cell plates were placed in trays and incubated 90 minutes in the incubator (37° C.). Thereafter the plates were incubated 30 minutes at room temperature, still protected from light.

Now the cell plates were ready to run in the Functional Drug Screening System (FDSS).

The assay buffer was HBSS with 20 mM HEPES, pH 7.4 and 3 mM CaCl₂.

### FDSS Ca Assay

200 mL 10 mM compound solution in DMSO was diluted in 50 μL assay buffer. The final test concentrations in the cell plates were 20-10-5-2.5-1.25-0.625-0.312-0.156-0.078-0.039 μM. Assay buffer and 3 μM PNU-120596 were used for control. The agonist acetylcholine was added to a final concentration of 20 μM (˜EC100). In the FDSS7000 the Ex480-Em540 was measured with 1 second intervals. The baseline was made of 5 frames before addition of test compounds, and 95 frames more were made before addition of acetylcholine. The measurement stopped 30 frames after the 2^{nd} addition. Raw data for each well were collected as “the maximum fluorescence count” in the interval 100-131 seconds and as “the average fluorescence count” in the interval 96-100 seconds. The positive allosteric modulation in the 2^{nd} addition was the enhancement of agonist response with test compound compared to agonist alone.

Results were calculated as % modulation of test compound compared to the reference PNU-120596 set to 100%. From these data EC₅₀ curves were generated giving EC₅₀, hill and maximum stimulation.

The compounds of the present invention characterized in the flux assay generally possess EC, values below 20.000 nM or less such as below 10.000 nM. Many compounds, in fact have EC₅₀ values below 5.000 nM. Table 1 shows EC₅₀ values for exemplified compounds of the invention.

TABLE 1

| Compound | EC₅₀(nM) |
|---|---|
| 1 | 3900 |
| 2 | 3600 |
| 3 | 4100 |
| 4 | 7100 |
| 5 | 3300 |
| 6 | 2500 |
| 7 | 2200 |
| 8 | 5100 |
| 9 | 4000 |
| 10 | 7200 |
| 11 | 2800 |
| 12 | 4900 |
| 13 | 7800 |
| 14 | 6300 |
| 15 | 6000 |
| 16 | 5500 |
| 17 | 11000 |
| 18 | 6500 |
| 19 | 3300 |
| 20 | 4600 |
| 21* | 20000 |
| 22* | 20000 |
| 23 | 3600 |
| 24* | 20000 |
| 25 | 7200 |
| 26 | 11000 |
| 27 | 5000 |
| 28 | 6500 |
| 29 | 5100 |
| 30 | 2700 |
| 31 | 5600 |
| 32 | 6200 |
| 33 | 8900 |
| 34* | 20000 |
| 35* | 20000 |
| 36* | 20000 |
| 37 | 9500 |

*Selected compounds of the invention were tested in the more comprehensive oocyte assay (Example 3). Compounds No. 21, 22, 24, 34, 35 and 36 were tested subsequently in the oocyte assay with the result of a positive NNR PAM activity in this assay.

### Example 3

### α7 NNR Oocyte Assay

Expression of α7 nACh Receptors in Xenopus Oocytes.

Oocytes were surgically removed from mature female Xenepus laevis anaesthetized in 0.4% MS-222 for 10-15 min. The oocytes were then digested at room temperature for 2-3 hours with 0.5 mg/mL collagenase (type IA Sigma-Aldrich) in OR2 buffer (82.5 mM NaCl, 2.0 mM KCl, 1.0 mM MgCl₂ and 5.0 mM HEPES, pH 7.6). Oocytes avoid of the follicle layer were selected and incubated for 24 hours in Modified Barth's Saline buffer (88 mM NaCl, 1 mM KCl, 15 mM HEPES, 2.4 mM NaHCO₃, 0.41 mM CaCl₂, 0.82 mM MgSO₄, 0.3 mM Ca(NO₃)₂) supplemented with 2 mM sodium pyruvate, 0.1 U/l penicillin and 0.1 μg/1 streptomycin. Stage 1V oocytes were identified and injected with 4.2-48 nl of nuclease free water containing 0.1-1.2 ng of cRNA coding for human α7 nACh receptors or 3.0-32 ng of cRNA coding for rat α7 nACh receptors and incubated at 18° C. for 1-10 days when they were used for electrophysiological recordings.

Electrophysiological Recordings of α7 nACh Receptors Expressed in Oocytes.

Oocytes were used for electrophysiological recordings 1-10 days after injection. Oocytes were placed in a 1 mL bath and perfused with Ringer buffer (115 mM NaCl, 2.5 mM KCl, 10 mM HEPES, 1.8 mM CaCl₂, 0.1 mM MgCl₂, pH 7.5)'. Cells were impaled with agar plugged 0.2-1 MΩ electrodes containing 3 M KCl and voltage clamped at −90 mV by a GeneClamp 500B amplifier. The experiments were performed at room temperature. Oocytes were continuously perfused with Ringer buffer and the drugs were applied in the perfusate. ACh (30 μM) applied for 30 sec were used as the standard agonist for activation of the α7 nACh receptors. In the standard screening set-up the new test compound (10 μM or 30 μM) were applied for 1 min of pre-application allowing for evaluation of agonistic activity followed by 30 sec of co-application with ACh (30 μM) allowing for evaluation of PAM activity. The response of co-application was compared to the agonistic response obtained with ACh alone. The drug induced effects on both the peak response and the total charge (AUC) response were calculated thus giving the effect of drug induced PAM activity as fold modulation of the control response. Table 2 indicates fold modulation of compounds No. 21, 22, 24, 34, 35 and 36 at determined at 10 μM.

For more elaborate studies doses-response curves can be performed for evaluation of max-fold modulation and EC₅₀ values for both peak and AUC responses.

TABLE 2

| | Fold modulation, | Fold modulation, |
|---|---|---|
| Compound | peak | AUC |
| 21 | 1.7 | 1.7 |
| 22 | 8.1 | 3.2 |
| 24 | 5.1 | 2.5 |
| 34 | 1.9 | 1.9 |
| 35 | 14 | 9.2 |
| 36 | 7.9 | 3.9 |

1. A compound according to formula [I] 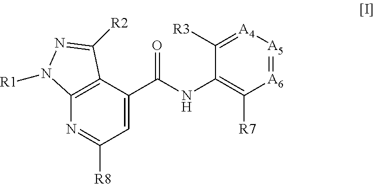
wherein A4 is C—R4 or N, A5 is C—R5 or N and A6 is C—R6 or N, provided that at least one of A4, A5 or A6 is N and no more than two of A4, A5 and A6 is N;
R1 is phenyl or heteroaryl; wherein said phenyl or heteroaryl is optionally substituted with one or more substituents R11, wherein each R11 is individually selected from C₁₋₆alkyl, halogen, hydroxy, haloC₁₋₆alkyl, C₁₋₆alkoxy, haloC₁₋₆alkoxy, cyano, C₁₋₆alkylsulfonyl, —S(O)₂NH₂ and —NR12R13, wherein R12 and R13 independently represent hydrogen or C₁₋₆alkyl;
R2 is selected from H, C₁₋₆alkyl, C₁₋₆alkenyl, C₂₋₆alkynyl, C₁₋₆alkoxy, halogen and cyano;
R3, R4, R5, R6 and R7 are selected independently of each other from H, C₁₋₆alkyl, C₂₋₆alkenyl, C₂₋₆alkynyl, C₁₋₆alkoxy, halogen, cyano, and —NR9R10, wherein R9 and R10 independently represent hydrogen, C₁₋₆alkyl, C₂₋₆alkenyl, C₂₋₆alkynyl, haloC₁₋₆alkyl or phenyl;
R8 is selected from H, C₁₋₆alkyl, C₂₋₆alkenyl, C₂₋₆alkynyl, C₁₋₆alkoxy, halogen, cyano and phenyl;
and pharmaceutically acceptable salts thereof.

2. The compound according to claim 1, wherein each R11 is individually selected from methyl, trifluoromethyl, fluorine, hydroxy, methoxy, difluoromethoxy, trifluoromethoxy, cyano, methylsulfonyl, —S(O)₂NH₂ and —N(CH₃)₂;
R2 is selected from H, methyl and methoxy;
R3, R4, R5, R6 and R7 are selected independently of each other from H, methyl, methoxy, cyano and N(CH₃)₂;
R8 is selected from H, methyl and phenyl.

3. The compound according to claim 1, wherein only one of A4, A5 or A5 is N.

4. The compound according to claim 1, wherein two of A4, A5 or A6 are N.

5. The compound according to claim 1, wherein R1 represents a phenyl optionally substituted with one or more R11.

6. The compound according to claim 1, wherein R1 represents a 6-membered heteroaryl optionally substituted with one or more R11.

7. The compound according to claim 1, wherein R1 represents a 5-membered heteroaryl optionally substituted with one or more R11.

8. The compound according to claim 1, wherein R1 is optionally substituted with one R11.

9. The compound according to claim 8, wherein said R11 is selected from methyl, trifluoromethyl, fluorine, hydroxy, methoxy, difluoromethoxy, trifluoromethoxy, cyano, methyl-sulfonyl, —S(O)₂NH₂ and —N(CH₃)₂.

10. The compound according to claim 1, wherein R2, R3, R4, R5, R6 and R7 are selected independently of each other from H, methyl and methoxy.

11. The compound according to claim 1, wherein R8 is selected from H, methyl and phenyl.

12. A compound according to claim 1 selected from
1: 1-(4-Methoxy-phenyl)-3,6-dimethyl-1H-pyrazolo[3,4-b]pyridine-4-carboxylic acid (2,4-dimethyl-pyridin-3-yl)-amide;
2: 3,6-Dimethyl-1-phenyl-1H-pyrazolo[3,4-b]pyridine-4-carboxylic acid (2,4-dimethyl-pyridin-3-yl)-amide;
3: 3,6-Dimethyl-1-phenyl-1H-pyrazolo[3,4-b]pyridine-4-carboxylic acid (3,5-dimethyl-pyridin-4-yl)-amide;
4: 1-(2-Methoxy-phenyl)-3,6-dimethyl-1H-pyrazolo[3,4-b]pyridine-4-carboxylic acid (2,4-dimethyl-pyridin-3-yl)-amide;
5: 1-(4-Methoxy-phenyl)-3,6-dimethyl-1H-pyrazolo[3,4-b]pyridine-4-carboxylic acid (2-methyl-pyridin-3-yl)-amide;
6: 3,6-Dimethyl-1-p-tolyl-1H-pyrazolo[3,4-b]pyridine-4-carboxylic acid (2,4-dimethyl-pyridin-3-yl)-amide;
7: 1-(4-Methoxy-phenyl)-3,6-dimethyl-1H-pyrazolo[3,4-b]pyridine-4-carboxylic acid (2-methoxy-4-methyl-pyridin-3-yl)-amide;
8: 1-(4-Methoxy-phenyl)-3,6-dimethyl-1H-pyrazolo[3,4-b]pyridine-4-carboxylic acid (4-methyl-pyridin-3-yl)-amide;
9: 1-(3-Methoxy-phenyl)-3,6-dimethyl-1H-pyrazolo[3,4-b]pyridine-4-carboxylic acid (2,4-dimethyl-pyridin-3-yl)-amide;
10: 3,6-Dimethyl-1-(6-methyl-pyridin-3-yl)-1H-pyrazolo[3,4-b]pyridine-4-carboxylic acid (2,4-dimethyl-pyridin-3-yl)-amide;
11: 1-(4-Methoxy-phenyl)-6-methyl-1H-pyrazolo[3,4-b]pyridine-4-carboxylic acid (2,4-dimethyl-pyridin-3-yl)-amide;
12: 1-(4-Methoxy-phenyl)-3,6-dimethyl-1H-pyrazolo[3,4-b]pyridine-4-carboxylic acid (4,6-dimethyl-pyrimidin-5-yl)-amide;
13: 1-(4-Fluoro-phenyl)-3,6-dimethyl-1H-pyrazolo[3,4-b]pyridine-4-carboxylic acid (2,4-dimethyl-pyridin-3-yl)-amide;
14: 1-(4-Cyano-phenyl)-3,6-dimethyl-1H-pyrazolo[3,4-b]pyridine-4-carboxylic acid (2,4-dimethyl-pyridin-3-yl)-amide;
15: 1-(4-Methoxy-phenyl)-3-methyl-1H-pyrazolo[3,4-b]pyridine-4-carboxylic acid (2,4-dimethyl-pyridin-3-yl)-amide;
16: 1-(6-Methoxy-pyridin-3-yl)-3,6-dimethyl-1H-pyrazolo[3,4-b]pyridine-4-carboxylic acid (2,4-dimethyl-pyridin-3-yl)-amide;
17: 3,6-Dimethyl-1-(4-trifluoromethoxy-phenyl)-1H-pyrazolo[3,4-b]pyridine-4-carboxylic acid (2,4-dimethyl-pyridin-3-yl)-amide;
18: 1-(4-Methoxy-phenyl)-3,6-dimethyl-1H-pyrazolo[3,4-b]pyridine-4-carboxylic acid (2,4,6-trimethyl-pyridin-3-yl)-amide;
19: 1-(4-Difluoromethoxy-phenyl)-3,6-dimethyl-1H-pyrazolo[3,4-b]pyridine-4-carboxylic acid
(2,4-dimethyl-pyridin-3-yl)-amide;
20: 1-(4-Methoxy-phenyl)-3,6-dimethyl-1H-pyrazolo[3,4-b]pyridine-4-carboxylic acid (4-methyl-pyrimidin-5-yl)-amide;
21: 3,6-Dimethyl-1-(4-sulfamoyl-phenyl)-1H-pyrazolo[3,4-b]pyridine-4-carboxylic acid (2,4-dimethyl-pyridin-3-yl)-amide;
22: 3,6-Dimethyl-1-(4-trifluoromethyl-phenyl)-1H-pyrazolo[3,4-b]pyridine-4-carboxylic acid (2,4-dimethyl-pyridin-3-yl)-amide;
23: 1-(4-Dimethylamino-phenyl)-3,6-dimethyl-1H-pyrazolo[3,4-b]pyridine-4-carboxylic acid (2,4-dimethyl-pyridin-3-yl)-amide;
24: 3-Methoxy-1-(4-methoxy-phenyl)-6-methyl-1H-pyrazolo[3,4-b]pyridine-4-carboxylic acid (2,4-dimethyl-pyridin-3-yl)-amide;
25: 3,6-Dimethyl-1-thiophen-3-yl-1H-pyrazolo[3,4-b]pyridine-4-carboxylic acid (2,4-dimethyl-pyridin-3-yl)-amide;
26: 3,6-Dimethyl-1-pyrimidin-5-yl-1H-pyrazolo[3,4-b]pyridine-4-carboxylic acid (2,4-dimethyl-pyridin-3-yl)-amide;
27: 3,6-Dimethyl-1-phenyl-1H-pyrazolo[3,4-b]pyridine-4-carboxylic acid (2-methoxy-4-methyl-pyridin-3-yl)-amide;
28: 3,6-Dimethyl-1-phenyl-1H-pyrazolo[3,4-b]pyridine-4-carboxylic acid (4-methoxy-2-methyl-pyridin-3-yl)-amide;
29: 3,6-Dimethyl-1-phenyl-1H-pyrazolo[3,4-b]pyridine-4-carboxylic acid (4-methoxy-6-Methyl-pyridin-3-yl)-amide;
30: 1-(4-Methoxy-phenyl)-3,6-dimethyl-1H-pyrazolo[3,4-b]pyridine-4-carboxylic acid (4-dimethylamino-pyridin-3-yl)-amide;
31: 3,6-Dimethyl-1-phenyl-1H-pyrazolo[3,4-b]pyridine-4-carboxylic acid (2-methyl-pyridin-3-yl)-amide;
32: 3,6-Dimethyl-1-phenyl-1H-pyrazolo[3,4-b]pyridine-4-carboxylic acid (4-methyl-pyridin-3-yl)-amide;
33: 1-(4-Methoxy-phenyl)-3,6-dimethyl-1H-pyrazolo[3,4-b]pyridine-4-carboxylic acid (4,6-dimethyl-pyridin-3-yl)-amide;
34: 1-(4-Methanesulfonyl-phenyl)-3,6-dimethyl-1H-pyrazolo[3,4-b]pyridine-4-carboxylic acid (2,4-dimethyl-pyridin-3-yl)-amide;
35: 1-(4-Methoxy-phenyl)-3-methyl-6-phenyl-1H-pyrazolo[3,4-b]pyridine-4-carboxylic acid (2,4-dimethyl-pyridin-3-yl)-amide;
36: 1-(4-Methoxy-phenyl)-3,6-dimethyl-1H-pyrazolo[3,4-b]pyridine-4-carboxylic acid (4-cyano-pyridin-3-yl)-amide;
37: 1-(4-Hydroxy-phenyl)-3,6-dimethyl-1H-pyrazolo[3,4-b]pyridine-4-carboxylic acid (2,4-dimethyl-pyridin-3-34)-amide;
and pharmaceutically acceptable salts of any of these compounds.

13. A method for the treatment of a disease or disorder which method comprises administering a therapeutically effective amount of a compound according to claim 1 to a patient in need thereof wherein the disease or disorder is selected from the group consisting of Psychosis; Schizophrenia; cognitive disorders; cognitive impairment associated with schizophrenia; Attention Deficit Hyperactivity Disorder (ADHD); autism spectrum disorders, Alzheimer's disease (AD); mild cognitive impairment (MCI); age associated memory impairment (AAMI); senile dementia; AIDS dementia; Pick's disease; dementia associated with Lewy bodies; dementia associated with Down's syndrome; Huntington's Disease; Parkinson's disease (PD); obsessive-compulsive disorder (OCD); traumatic brain injury; epilepsy; post-traumatic stress; Wernicke-Korsakoff syndrome (WKS); post-traumatic amnesia; cognitive deficits associated with depression; diabetes, weight control, inflammatory disorders, reduced angiogenesis; amyotrophic lateral sclerosis and pain.

14. A pharmaceutical composition comprising a compound according to claim 1 and one or more pharmaceutically acceptable carriers or excipients.
